(19) United States
(12) Patent Application Publication
(10) Pub. No.: US 2009/0214654 A1
(43) Pub. Date: Aug. 27, 2009

(54) TREATMENT OF ANEURYSM WITH APPLICATION OF CONNECTIVE TISSUE STABILIZATION AGENT IN COMBINATION WITH A DELIVERY VEHICLE

(76) Inventors: Jason C. Isenburg, Victoria, MN (US); Narendra R. Vyavahare, Easley, SC (US); Matthew F. Ogle, Fitchburg, WI (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 12/390,156

(22) Filed: Feb. 20, 2009

Related U.S. Application Data
(60) Provisional application No. 61/066,688, filed on Feb. 21, 2008.

(51) Int. Cl. A61K 9/12; A61P 9/00; A61K 31/352; A61K 31/7034; A61K 31/11

(52) U.S. Cl. 424/488; 424/484; 514/456; 514/25; 514/705

(57) ABSTRACT

Delivery vehicles for controlled release of connective tissue stabilization agent for the treatment of vascular aneurysms are described. The delivery vehicle generally is combined with a connective tissue stabilization agent to form a therapeutic composition. The treatment of an aneurysm can be achieved through release of connective tissue stabilization agent from the delivery vehicle to the aneurysm. The connective tissue stabilization agent can be collagen stabilization agent, elastin stabilization agent, or a combination thereof. The aneurysm can be treated individually, simultaneously or sequentially with collagen stabilization agent and elastin stabilization agent embedded in separate delivery vehicles.

### CROSS REFERENCE TO RELATED APPLICATIONS

This application claims priority to U.S. provisional patent application Ser. No. 61/066,688, filed on Feb. 21, 2008 to Isenburg et al., entitled “Treatment of Aneurysm with Application of Elastin Stabilizing Agent Embedded in a Delivery System,” incorporated herein by reference.

### FIELD OF THE INVENTION

The inventions, in general, are related to a delivery vehicle as a part of a therapeutic composition to treat vascular aneurysm. The inventions are further related to methods of making and using the delivery vehicle.

### BACKGROUND

Aneurysms may be caused by a variety of mechanisms including atherosclerotic disease, defects in arterial components, genetic susceptibilities, high blood pressure, and others. In particular, abdominal aortic aneurysms (AAAs) are degenerative diseases characterized by destruction of arterial architecture and subsequent dilatation that may eventually lead to fatal ruptures. AAAs as well as other aneurysms are a serious health concern, specifically for the aging population. Currently the sole approved treatment of AAA is surgical replacement of the diseased artery or endovascular stent graft repair. Although often effective, these surgical options are not without their own drawbacks. For instance, endovascular stents are anatomically appropriate for only 30% to 60% of AAA patients at the outset and present the risk of endoleaks and graft displacement. Moreover, open surgery for full-size graft insertion is highly invasive, limiting its use to those patients that can tolerate high operative risk. Early diagnosis and treatment of aneurysmal disease therefore are unmet clinical needs that are yet to be addressed.

### SUMMARY OF THE INVENTION

In a first aspect, the invention relates to a therapeutic composition for treatment of aneurysm in a patient. The therapeutic composition comprises a connective tissue stabilization agent in combination with a delivery vehicle. The delivery vehicle comprises a hydrogel, nanoparticles, or a combination thereof. In one embodiment, the hydrogel of the delivery vehicle comprises penta-galloylglucose in a gel form. In some embodiments, the hydrogel comprises Pluronic™ hydrogel. In one embodiment, the hydrogel, the nanoparticle, or both is or are loaded with penta-galloylglucose, glutaraldehyde, or a combination thereof. In one embodiment, the nanoparticles comprises poly (lactic acid-co-glycolic) acid. In one embodiment, the hydrogel comprises Pluronic™ F-127 hydrogel.

The connective tissue stabilization agent of the therapeutic composition comprises an elastin stabilization agent, a collagen stabilization agent, or a combination thereof. The elastin stabilization agent comprises a hydrophobic region and a plurality of functional groups capable of hydrogen bonding. In some embodiments, the elastin stabilization agent comprises tannic acid or a derivative thereof, a flavonoid or a flavonoid derivative, a flavolignan or a flavolignan derivative, a phenolic rhizome or a phenolic rhizome derivative, a flavan-3-ol or a flavan-3-ol derivative, an ellagic acid or an ellagic acid derivative, a procyanidin or a procyanidin derivative, anthocyanins, quercetin, (+)-catechin, (−)epicatechin, pentagalloylglucose, nobotaiun, epigallocatechin gallate, gallotannins, an extract of olive oil or a derivative of an extract of olive oil, cocoa bean or a derivative of a cocoa bean, camellia or a derivative of camellia, licorice or a derivative of licorice, sea whip or a derivative of sea whip, aloe vera or a derivative of aloe vera, chamomile or a derivative of chamomile, a combination thereof, or a pharmaceutically acceptable salt thereof. The collagen stabilization agent comprises a cross-linker of functional groups in collagen. In some embodiments, the collagen stabilization agent comprises glutaraldehyde, diamine, genipin, acyl azide, epoxyamine, a combination thereof, or a pharmaceutically acceptable salt thereof. In one embodiment, the connective tissue stabilization agent further comprises gallic acid scavenger, a lipid lowering medication, an anti-bacterial agent, an anti-fungal agent, or a combination thereof.

In a second aspect, the invention relates to a method of making a therapeutic composition for treatment of aneurysm in a patient. The method comprises combining a connective tissue stabilization agent with a delivery vehicle to form the therapeutic composition so the connective tissue stabilization agent is released over a period of time to the aneurysm upon contact with bodily fluids. In some embodiments, the combining step comprises forming a solution of precursor of the hydrogel and the connective tissue stabilization agent. In one embodiment, the combining step comprises forming a solution of Pluronic™ block copolymers with penta-galloylglucose, glutaraldehyde, or a combination thereof. In some embodiments, the combining step comprises embedding the connective tissue stabilization agent into nanoparticles. In one embodiment, the connective tissue stabilization agent is embedded inside nanoparticles using emulsion solvent evaporation technique. In some embodiments, the combining step further comprises adding the connective tissue stabilization agent embedded nanoparticles into hydrogels to form the controlled release therapeutic composition. In one embodiment, the combining step comprises forming a dispersion of Pluronic™ block copolymers with penta-galloylglucose-loaded poly(lactic acid-co-glycolic) acid nanoparticles with optional addition of glutaraldehyde-loaded poly(lactic acid-co-glycolic) acid nanoparticles. In some embodiments, the therapeutic composition further comprises pharmaceutically acceptable carriers and/or excipients.

In a third aspect, the invention relates to a method of using a therapeutic composition for the treatment of aneurysm in a patient. The method comprises applying the therapeutic composition to the aneurysm. The therapeutic composition comprises a connective tissue stabilization agent with a delivery vehicle, the connective tissue stabilization agent being released over a period of time to the aneurysm. The therapeutic composition can be applied intravascular, perivascularly, or a combination thereof to the aneurysm. In some embodiments, the treatment method comprises isolating the aneurysm from within a blood vessel using a device placed within the blood vessel and aspirating the isolated aneurysm before the application of the therapeutic composition using the device. In one embodiment, the therapeutic composition is applied to the aneurysm through a perivascular wrap. In some embodiments, the treatment method is applied plurality of times to the aneurysm in the patient.

In a fourth aspect, the invention relates to a method for treatment of aneurysm in a patient by applying connective tissue stabilization agent in the form of a hydrogel, nanoparticles, or a combination thereof to the aneurysm. In one embodiment, the connective tissue stabilization agent is pentagalloylglucose, epigallocatechin gallate, or a combination thereof.

In a fifth aspect, the invention relates to an active agent delivery vehicle that comprises a hydrogel and nanoparticles dispersed within the hydrogel. The nanoparticles comprise the active agent and a bioresorbable polymer binder.

### BRIEF DESCRIPTION OF THE DRAWINGS

FIG. 1 is a graphic illustration of abdominal aorta aneurysm (AAA).

FIG. 2 is graphic illustration of surgical treatment options for AAAs: (A) endovascular stent graft repair; (B) open surgical repair/replacement; and (C) perivascular girdle wrap.

FIG. 3 is the chemical structure of penta-galloylglucose (PGG).

FIG. 4 is (Top) a schematic side view of a delivery device for delivery of the therapeutic composition described herein and (Bottom) a cross sectional view of the shaft of the delivery device.

FIG. 5 is schematic illustration of the delivery device of FIG. 4 placed inside a vessel, isolating and aspirating an aneurysm.

FIG. 6 is (Top) a diagram showing cumulative binding of tannic acid (TA) to pure aortic elastin and (Bottom) a representative schematic diagram of interactions between TA and elastin.

FIG. 7 is (Left) a diagram showing tannic acid mediated stabilization of pure elastin against the action of elastase and (Right) histologies of fresh porcine aorta (A), pure aortic elastin (B), aortic elastin exposed to elastase (C), and elastin stabilized with TA (D).

FIG. 8 is a diagram showing the protective efficacy of TA and PGG as elastin stabilizing agents.

FIG. 9 is a flow diagram illustrating the concept of how to estimate PGG's protective effect using artificially partially digested elastin.

FIG. 10 is a diagram showing changes in dry tissue weights after the second round of elastase treatment with respect to the dry weights collected after the first round of elastase treatment in control (saline) and PGG treated (only before second round) samples.

FIG. 11 is a diagram showing the mean percent change in diameter of abdominal aorta at 28 days relative to day 0 in rats.

FIG. 12 is (Left) a diagram showing the result from desmosine analysis performed on non-surgery control rat aorta (day 0) compared to aorta collected 28 days after chemical injury of PGG treated and saline-treated groups and (Right) histology of the same aorta samples.

FIG. 13 is a diagram, a table and histologies showing the delivery of PGG to aneurysmal aorta prevents AAA progression in rats.

FIG. 14 is a plot of the percentage digestions of portions of porcine carotid arteries treated with different therapeutic compositions showing varied abilities to resist elastase digestion.

FIG. 15 is a plot of stress versus strain of portions of porcine carotid arteries treated with different therapeutic compositions showing varied uniaxial tensile strength.

FIG. 16(A) is a photograph showing a perspective view of a porcine aorta cut transversely into ring segments and a photograph of the top view of the ring segment being cut open.

FIG. 16(B) is a set of photographs of the treated ring segments that were cut open and allowed to relax following various treatments of the tissue and how the opening angle of aortic ling is measured.

FIG. 16(C) is a plot of the opening angles of aortic rings compared for different treatments.

FIG. 17 is a plot of the percentage digestion of treated tissues compared for different treatments of the tissue following exposure to collagenase.

FIG. 18 is a schematic flow diagram illustrating the concept of how in vitro preparation and characterization of PGG-polymer formulations followed by in vivo evaluation of PGG delivery can be preformed.

FIG. 19 is a schematic diagram of a proposed animal experiment to evaluate the effectiveness of the treatment described herein, showing in vivo application of PGG to aneurysmal rat aorta and subsequent analysis thereof.

FIG. 20 is a photograph showing rat aorta retrieval and preparation.

### DETAILED DESCRIPTION OF PREFERRED EMBODIMENTS

The delivery vehicles described herein provide controlled release of one or more connective tissue stabilization agents to aneurysm to improve the efficacy of the stabilization agents and provide for desirable delivery approaches. The description herein additionally provide methods of making the delivery vehicles and methods of treatment of aneurysm with intravascular or perivascular application of connective tissue stabilizing agent embedded in and/or associated with the delivery vehicle, such as a Pluronic™ hydrogel and/or polymeric nanoparticles. The therapeutic compositions formed by the combination of the stabilization agents with the delivery vehicles can be delivered to an aneurysm at either the exterior or interior of a blood vessel. While the description herein focuses on aortic aneurysms, the treatment approaches can be generalized to other aneurysms based on the teachings herein.

Some embodiments of stabilization agents and devices used for the treatment of aneurysms and diagnostic biomarkers are described in U.S. Pat. No. 7,252,834 (the '834 Patent) to Vyavahare et al., entitled “Elastin Stabilization of Connective Tissue”, U.S. Provisional Patent Application 61/113,881 (the '881 Application) to Isenburg et al., entitled “Compositions for Tissue Stabilization”, U.S. patent application Ser. No. 12/173,726 (the '726 Application) to Ogle et al., entitled “Devices for the Treatment of Vascular Aneurysm”, and U.S. patent application Ser. No. 12/355,384 (the '384 Application) to Ogle et al., entitled “Diagnostic Biomarkers for Vascular Aneurysm”, all of which incorporated herein by reference. The methods and compositions herein provide in some embodiments for refinement of the treatment methods and therapeutic compositions in the '834 patent and the '881 Application. The examples below focus on using penta-galloylglucose (PGG) as elastin stabilizing agent and glutaraldehyde (GLU) as collagen stabilizing agent. Other connective tissue stabilizing agents, some of which are noted below, can be similarly released in a controlled and time release way based on the description herein.

In some embodiments, the therapeutic formulations described herein comprise one or more tissue stabilization agents combined with a delivery vehicle. The delivery vehicle can be a hydrogel polymer. A hydrogel polymer provides for the gradual release of the stabilization agent as well as a more controlled delivery of the agent to the aneurysm. Also, the stabilization agents can be provided within polymer nanoparticles. The nanoparticles provide for the controlled release of the tissue stabilization agents to the aneurysm. Furthermore, there can be farther advantages with respect to combining the nanoparticles infused with the stabilization agents within a hydrogel.

Delivery approaches such as those described in the '726 Application have been developed that provide for the local delivery of the therapeutic compositions at the aneurysm. The use of the delivery vehicles herein provide for the sustained release of tissue stabilization agents at aneurysm over a period of time. This gradual release provides for the treatment of the aneurysmal tissue with a concentration of the stabilization agents that varies less over time for a more predictable therapeutic effect. Also, the properties of the delivery vehicle can be selected to provide for a corresponding efficacy of the stabilization agents with respect to aneurysmal tissue stabilization. In general, an effective amount of the therapeutic composition used for aneurysm treatment is determined by measurable stabilization of the aneurysmal tissue such as those exemplified in the examples discuss below.

### Aneurysms and Clinical Management

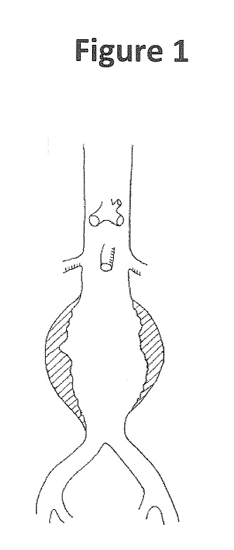

Aneurysms are abnormal widening or ballooning of a portion of an artery, related to structural weakness in the wall of the blood vessel such as the abdominal aorta aneurysm (AAA) shown in FIG. 1. Some common locations for aneurysms include the abdominal aorta, (abdominal aortic aneurysm, AAA), thoracic aorta, and brain arteries. Aneurysms grow over a period of years and pose great risks to health. Aneurysms have the potential to dissect or rupture, causing massive bleeding, stroke, and hemorrhagic shock, which can be fatal in more than 80% of cases. AAAs are a serious health concern, specifically for the aging population, being among the top ten causes of death for patients older than 50. The estimated incidence for abdominal aortic aneurysm is about 50 in every 100,000 persons per year. Approximately 60,000 operations are performed each year in the U.S. for abdominal aortic aneurysms alone. In children, AAA can result from blunt abdominal injury or from Marfan's syndrome, an elastic fiber defect in major arterial walls, such as the aorta.

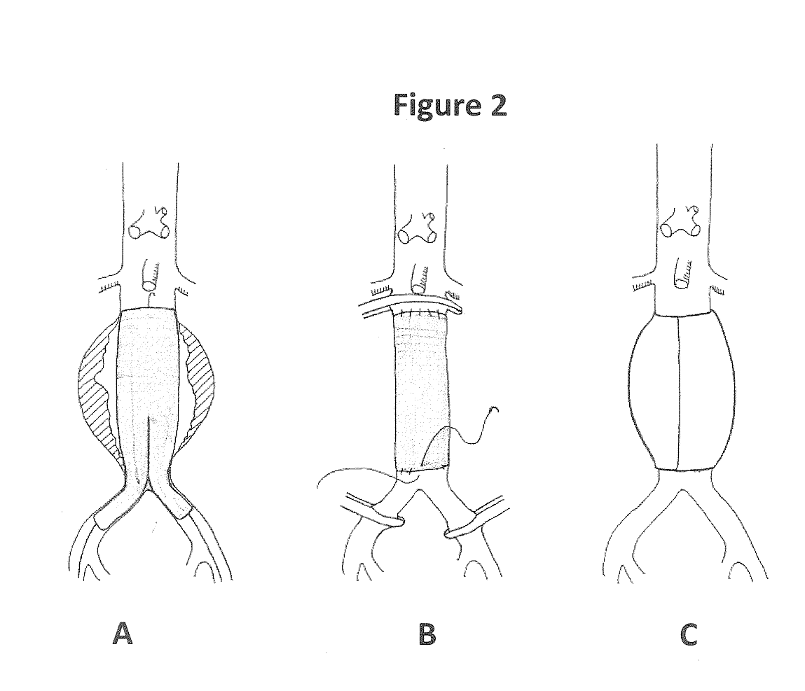

Methods for diagnosing and identifying the degree of aneurysm expansion are available due to developments in high resolution imaging technology (CT, MRI). After initial diagnosis of a small aneurysm (larger than 2 cm in diameter), the most common medical approach is to periodically monitor its development (for instance, every 6 months) and if it reaches a certain stage (typically larger than 5.5 cm diameter), to apply surgical treatment. This involves endovascular stent graft repair (placement of a tube inside the vessel) or complete replacement of the diseased aorta with an artificial mesh vascular graft, as shown in FIGS. 2A and 2B, respectively. Surgical treatment of aneurysms saves thousands of lives every year and improves quality of life. However, survival rates can drop to only 50% at 10 years postoperative due to surgery-related complications or device-related problems. In addition, endovascular stents are anatomically appropriate for only 30% to 60% of AAA patients at the outset and present the risk of endoleaks and graft displacement. Moreover, open surgery for full-size graft insertion is highly invasive, limiting its use to those patients that can tolerate high operative risk. Treatment options are particularly limited for patients with small or moderate aneurysms, a group which makes up the largest percentage of all AAA patients. Consequently, novel therapeutic approaches targeted at hindering the progression of AAAs promptly after diagnosis would be extremely beneficial for aneurysm patients.

For many patients who have a small or early stage aneurysm, there is unfortunately no current option or therapy. In these cases, the aortic diameter is periodically monitored until it reaches a critical threshold (typically 5.5 cm), at which point surgical repair or replacement is preformed as described previously. This “wait and see” approach is not without risk, however, as it has been estimated that as many as 10% of the abdominal aortic aneurysms that rupture do so at diameters less than 5 cm. Therefore, alternative treatments targeted at limiting aortic expansion such as by stabilizing tissue components such as elastin and collagen may be helpful in reducing incidence of rupture and circumventing the need for surgical repair.

Recent techniques have been developed for early detection as well as to track the progress of aneurysm using a laboratory test, such as a blood test, a urine test or a combination thereof. Early detection techniques are described, for example, in copending U.S. patent application Ser. No. 12/355,384, filed on Jan. 16, 2009 to Ogle et al., entitled “Diagnostic Biomarkers for Vascular Aneurysm”, incorporated herein by reference. Connective tissue degradation products associated with aneurysmal tissue and enzymes associated with tissue degradation have been found to be useful as diagnostic biomarkers. The biomarkers can include, for example, elastin degradation product such as desmosine, isodesmosine and elastin degradation peptides, collagen degradation product such as pyridinoline, deoxypyridinoline, pro-collagen-IIIN terminal propeptides and N-telopeptides of type I collagen, degradation enzymes such as matrix metalloproteinase 1, 2, 8, 9, 12, 13, and 18, or a combination thereof. The diagnostic biomarkers offer a convenient and cost effective method for early diagnosis of aneurysm, thus presenting treatment opportunity and early intervention procedures with therapeutic compositions such as those described herein.

Elastin and collagen stabilization compositions and methods such as those described in U.S. Pat. No. 7,252,834 (the '834 Patent) to Vyavahare et al., entitled “Elastin Stabilization of Connective Tissue” and in U.S. Provisional Patent Application 61/113,881 (the '881 Application) to Isenburg et al., entitled “Compositions for Tissue Stabilization”, respectively have been developed as pharmacological alternative to surgery for treating aneurysm. Such pharmacological alternative addresses especially the unmet clinical need for treatment of early and moderate stage aneurysms. The formulations described herein provide improved delivery options for pharmacological treatments. The treatment can be achieved for example by using devices disclosed in U.S. patent application Ser. No. 12/173,726 (the '726 Application) to Ogle et al., entitled “Devices for the Treatment of Vascular Aneurysm,” incorporated herein by reference and described further below. The methods and delivery vehicle disclosed herein provide controlled release of the stabilization agents in the '834 patent and the '881 Application to make the therapeutic composition that is delivered to the aneurysm using the delivery device of the '726 Application.

The methods and compositions disclosed herein provide treatment options for early and moderate aneurysms that are normally not treated by surgical intervention. Early detection and treatment provides the opportunity for limiting the progression of the disease and subsequent danger, improving the quality of life of the aneurysm patient and lowering the cost relative to circumstances when the aneurysm is not treated until a late stage. The methods and compositions described herein additionally provide treatment possibilities for conditions where surgical intervention is not applicable, such as aneurysm in deep tissue. The combination of diagnosis, device, and therapeutic compositions provide life saving/change alternatives, which can be effectively applied at early and moderate stages of the disease to reduce patient suffering as well as to reduce societal costs.

Connective Tissue Degeneration within Aneurysms and Connective Tissue Stabilization

Connective tissue is the framework upon which the other types of tissue, i.e., epithelial, muscle, and nervous tissues, are supported. There are many specialized types of connective tissue, one example being artery. In many cases, the characteristics of aneurysms are degeneration of arterial structural proteins including elastin and collagen, inflammatory infiltrates, calcification, and overall destruction of arterial architecture. This results in loss of mechanical properties and progressive dilatation of the artery. Severe elastin degradation is reported within these aneurysmal tissues, as evidenced by heavy degeneration of the arterial architecture, decreased medial elastin content, and disrupted or fragmented elastic lamellae. This degradation is particularly significant when one considers the inability of elastin to promptly revitalize itself (as evidenced by its nearly 70-year biological half-life), unlike some other relatively dynamic matrix components. Furthermore, degradation of elastin results in the release of soluble elastin peptides that are active in protease up regulation, chemotaxis, cellular proliferation, and various other biological activities. The extreme bioactivity of elastin peptides underscores the clinical significance of elastin degradation within aneurysmal tissues and the subsequent need to protect elastin from degeneration.

Additionally, collagen is present throughout the aneurysm tissue. See, for example, Loftus I M, Thompson M M. Vasc Med 2002; 7(2): 117-133, incorporated herein by reference. In the course of aneurysm development, it has been suggested that the processes of degradation and regeneration of collagen alternates. Once the collagen degradation reaches a particular degree, the rupture of the aneurysm tissue may occur. See, for example, Choke E, Cockerill G, Wilson W R, et al. Eur J Vase Endovasc Surg 2005; 30(3): 227-244, incorporated herein by reference. Stabilization of collagen in aneurysm tissue can be an effective aspect for treating vessel damage associated with an aneurysm.

As described above in the '834 patent, degradation of connective tissue can be prevented or slowed through the stabilization of the elastin component of the tissue with a phenolic compound. In particular, it is believed that any of a number of natural and synthetic phenolic compounds can bind elastin and thereby protect elastin from degradation, for instance due to the action of elastin degrading enzymes. In some embodiments, elastin stabilizing phenolic compounds include, for example, any compound that comprises at least one phenolic group bound to a hydrophobic core. While not wishing to be bound by any particular theory, it is believed that interaction between the phenolic compound and elastin proteins have aspects involving both the hydroxyl group as well as the hydrophobic core of the molecules. In certain embodiments, the phenolic compounds can comprise one or more double bonds, with which the phenolic compounds can covalently bind to the elastin, forming an even stronger and more permanent protective association between the phenolic compound and the elastin of the connective tissue. In addition, the large hydrophobic regions of the elastin protein, which are believed to contain sites susceptible to elastase-mediated cleavage, are also believed to contain sites of association between the hydrophobic core of the phenolic compound and the protein. Thus, the association between the phenolic compound and the protein molecules are believed to protect specific binding sites on the protein targeted by enzymes through the association of the protein with the hydrophobic core and can also sterically hinder the degradation of the protein through the development of the large three dimensional cross-link structures.

Phenolic compounds in some embodiments can comprise a hydrophobic core and one or more phenol groups extending from the hydrophobic core of the molecule. For instance, exemplary phenolic compounds can include, but are not limited to, flavonoids and their derivatives (e.g., anthocyanins, quercetin), flavolignans, phenolic rhizomes, flavan-3-ols including (+)-catechin and (−)-epicatechin, other tannins and derivatives thereof (such as tannic acid, pentagalloylglucose, nobotanin, epigallocatechin gallate, and gallotannins), ellagic acid, procyanidins, and the like.

Phenolic compounds include synthetic and natural phenolic compounds. For example, natural phenolic compounds can include those found in extracts from natural plant-based sources such as extracts of olive oil (e.g., hydroxytyrosol(3,4-dihydroxyphenylethanol) and oleuropein, extracts of cocoa bean that can contain epicatechin and analogous compounds, extracts of Camellia including C. senensis (green tea) and C. assaimic, extracts of licorice, sea whip, aloe vera, chamomile, and the like.

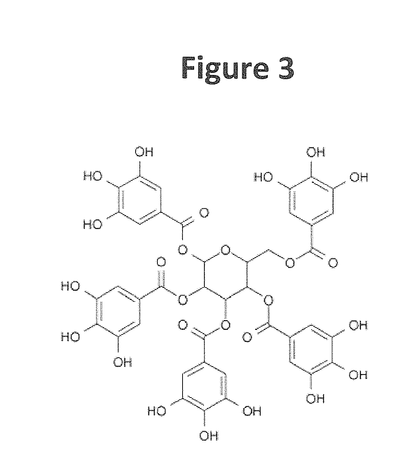

In one embodiment, the phenolic compounds can be tannins and derivatives thereof. Tannins can be found in many plant species. For example, the tea plant (Camellia sinensis) has a naturally high tannin content. Green tea leaves are a major plant source of tannins, as they not only contain the tannic and gallic acid groups, but also prodelphinidin, a proanthocyanidin. Tainins are also found in wine, particularly red wine as well as in grape skins and seeds. Pomegranates also contain a diverse array of tannins, particularly hydrolysable tannins. pentagalloylglucose (PGG) and tannic acid (TA) are members of the tannin family, a group of naturally derived polyphenolic compounds. PGG is a less toxic derivative of tannic acid. PGG is naturally occurring, relatively non-toxic and not expected to exhibit significant side effects. PGG, chemical structure shown in FIG. 3 is characterized by a D-glucose molecule esterified at all five hydroxyl moieties by gallic acid(3,4,5-trihydroxybenzoic acid). Periarterial treatment with PGG preserves elastin fiber integrity and hinders aneurysmal dilatation of the abdominal aorta in a clinically relevant model of AAA. In general, it is understood that the PGG molecule can have 1-4 galloyl group(s) and the galloyl groups can assume different stereo chemical forms. For example, PGG can be in either alpha or beta forms.

Additionally, collagen crosslinking/stabilization compositions have been found to provide a high degree of stabilization of vascular tissue associated with aneurysms and other degeneration of blood vessels in copending U.S. provisional patent application Ser. No. 61/113,881 to Ogle et al., entitled “Compositions for Tissue Stabilization,” incorporated herein by reference. In some embodiments, the collagen crosslinking/stabilization agent can be effectively combined with an elastin stabilizing agent. The treatment agents can be contacted with the tissue simultaneously or sequentially.

Multi-functional reagents, such as glutaraldehyde, diamine, genipin, acyl azide, and epoxyamine, are known to cross-link functional groups in collagen thereby stabilize collagen and tissue having a collagen component. Some known functional groups for collagen cross-linking are amino, thiol, hydroxyl, and carbonyl in collagen and/or nearby proteins. By binding to and crosslinking collagen and/or nearby proteins, the multi-functional agents can increase the mechanical strength of the tissue. In the case of aneurysm, the increased mechanical strength of aneurysm vessel can correspondingly increase the tolerance of the treated aneurysm tissue to burst pressure, thus decrease the risk of rupture of the vessel. Tissue treated with collagen crosslinking/stabilization agent with or without combination with elastin stabilization agent may exhibit enhanced mechanical property, resistance to enzymatic degradation such as elastase and collagenase, and high thermal denaturation temperature as shown in Examples 7-11.

Some collagen stabilization agent maybe used for effective in vivo treatment employing a delivery device followed by additional treatment with elastin stabilization agent. Agents may have acute in vivo toxicity such that isolation of the treatment site during the delivery and treatment process can be advantageous. Some collagen stabilization agents maybe used for slow release at the site of the aneurysm, for example, in the form of coating of a stent, embedded in surgical girdle that wraps around the aneurysm vessel, or in delivery vehicles described herein.

Glutaraldehyde and other multi-functional aldehyde compounds are known to bind to and stabilize collagen in the wall of a blood vessel. Glutaraldehyde in particular self-polymerizes to form polymer chains that are believed to be effective at crosslinking between collagen fibers. Glutaraldehyde polymerizes with itself and/or with nearby active groups from collagen and/or other proteins creating crosslinks in the treated tissue. The chemical crosslinks in the tissue can contribute to increased resistance to degradation of the treated tissue. However, residual unreacted free aldehyde groups from glutaraldehyde can contribute with regards to toxicity and calcification. Treatment of bioprosthetic tissue to reduce toxicity is described in U.S. Pat. No. 6,471,723 to Ashworth et al., entitled “Biocompatible Prosthetic Tissue,” incorporated herein by reference.

By binding to and crosslinking collagen, glutaraldehyde increases the mechanical strength of the tissue. The in vivo application of the glutaraldehyde alone and in combination with PGG have been briefly discussed in the '834 patent and the '881 Application with respect to treatment of aneurysms. For in vivo treatment at the site of the aneurysm inside a blood vessel, however, the amount of glutaraldehyde, treatment concentration, treatment time, and application of toxicity control agent(s) can be selected to achieve desired treatment effects while avoiding undesirable effects from excessive treatment, such as excessive cellular toxicity and over-stiffening of the vessel well. Preliminary experimental results using glutaraldehyde and/or an elastin stabilizer such as PGG or tannic acid have been presented and discussed in further detail in Examples 7-11.

One of the alternative collagen stabilizing agents comprises diamines, generally with at least two free primary amine groups, such as 1,6-hexanediamine and 1,7-heptanediamine. The diamines bond to carboxyl groups in proteins to form a crosslinked structure. It has been found that coupling agents and coupling enhancers facilitate this crosslinking/stabilization process with diamines. For example, suitable coupling agents include carbodiimides, such as 1-ethyl-3-(3-dimethyl aminopropyl)-carbodiimide (EDC) and/or N-hydroxysuccinimide (NHS). The carbodiimides function as a coupling agent in the crosslinking/stabilization reaction, and are generally used along with a coupling enhancer. For example, EDC can be used in conjunction with N-hydroxysulfosuccinimide (Sulfo-NHS), which acts as an enhancer to the reaction. Other suitable coupling enhancers include, for example, N-hydroxybenzotriazole (HOBt), N,N-dimethyl-4-aminopyridine (DMAP) and N-hydroxysuccinimide. By coupling the amine and carboxyl groups within the tissue, this treatment creates amide bonds or bridges between and/or inside proteins, thus crosslinking the tissue. In vitro crosslinking of bioprosthetic tissue with diamines along with coupling agents and/or coupling enhancers is described further in published U.S. patent application 2006/0159641A to Girardot et al., entitled “Variably Crosslinked Tissue,” incorporated herein by reference.

Collagen stabilization can be achieved using other active agent or alternative methods. For example, collagen stabilization in tissue can be triggered by a light sensitive dye, similar to the PhotoFix™ technology used by Carbomedics for bioprosthetic heart valves; genipin is a naturally occurring plant compound capable of crosslinking collagen; epoxy compounds have reactive functional groups that are reactive with several functional groups found in proteins, such epoxies can be used to crosslink proteins, especially collagen, within tissue. Additionally, epoxy amine polymer compounds are also suitable collagen crosslinking agents that are described further in U.S. Pat. No. 6,391,538 to Vyavahare et al., entitled “Stabilization of Implantable Bioprosthetic Tissue,” incorporated herein by reference. An example of a poly-epoxyamine compound suitable as a collagen crosslinking agent is triglycidylamine, a triepoxy amine. Moreover, free carboxyl groups on collagen can be converted into acyl azide groups, which react with free amino groups on adjacent side chains to crosslink the collagen tissue. This crosslinking approach is described in Petite et al. Biomaterials 1995; 16(13): 1003-1008, incorporated herein by reference.

In general, connective tissue targeted with the therapeutic agent(s) or composition(s) can be stabilized so as to be less susceptible to protein degradation as well as having improved mechanical strength to resist distortion of the natural shape and possible bursting. In some embodiments, the collagen crosslinking/stabilization agents can be administered alone. In other embodiments, the collagen crosslinking/stabilization agents can be combined with elastin stabilization agent. In yet other embodiments, the collagen crosslinking/stabilization agent and elastin stabilization agent can be administered in separate application steps sequentially to the site of aneurysm. The collagen crosslinking/stabilization agent and elastin stabilization agent can each have an appropriate application time, composition, delivery vehicle, and concentration. The treatment parameters such as concentration, composition, delivery vehicle, application device and method of delivery can be adjusted to suit variety of needs with respect to stabilizing tissues with collagen and/or elastin component.

### Delivery Vehicles and Therapeutic Composition

The therapeutic compositions of particular interest comprise one or more delivery vehicles combined with a tissue stabilization agent that is effective to stabilize connective tissue at an aneurysm. The delivery vehicles can be selected to provide a sustained release of the stabilization agent(s) as well as to control the conditions of the contact between the stabilization agent and the tissue. Suitable delivery vehicles can include, for example, a gel formed from a stabilization agent, a hydrogel composition, nanoparticles incorporating the stabilization agent or combinations thereof. Specifically, a particular effective therapeutic composition can be formulated by incorporating the stabilization agent(s) into nanoparticles that are then incorporated into a hydrogel. In some embodiments, the therapeutic compositions can be administered on multiple occasions to achieve the desire therapeutic effect. The length of the period between each administration can be determined by the combination of the specific release profile of the therapeutic composition used and the condition of the aneurysm. Throughout the treatment periods, diagnostic methods such as the diagnostic biomarkers disclosed in the '384 Application can be employed to monitor the condition of the aneurysm. The delivery vehicles disclosed herein can be similarly adapted to control release of any active agent of interest.

The sustained release disclosed herein is alternatively referred to as controlled release, which refers to continual delivery of the stabilization agent in vivo over a period of time following administration. Controlled release of the stabilization agent can be demonstrated by, for example, the continued therapeutic effect of the agent over time. Alternatively, controlled release of the agent may be demonstrated by detecting the presence of the agent in vivo over time. Prophetic examples below outline procedures to demonstrate in vitro and in vivo release profiles of PGG-loaded polymers. In some embodiments, the controlled release is less than about a week and can be less than four days. However, it is also contemplated that the controlled release can be for periods longer than one week using the composition. In some embodiment the release period can be about 1 hour, 2, hours, 4 hours, 8 hours, 12 hours, 24 hours, 48 hours, 72 hours, 96 hours, or a combination thereof. In some other embodiment, the release period is longer than about 5 weeks, 10 weeks, 15 weeks, 20 weeks, 25 weeks, 30 weeks, 35 weeks, 40 weeks, 45 weeks, 50 weeks or 55 weeks. In one embodiment, the release period is about 26 weeks. A person of ordinary skill in the art will recognize that additional ranges of time within these explicit ranges are contemplated and are within the present disclosure.

In some embodiments, a hydrogel is formed in vivo from a precursor of the hydrogel, such as block copolymers that crosslink when a threshold temperature such as human physiological temperature is reached. The hydrogel formed does not dissolve in aqueous solution generally as a result of crosslinking if the temperature remains about the same or higher. The block copolymers used are soluble at lower temperature such as room temperature. Because of the thermo-gelation properties of the block copolymers, tissue stabilization agent can be combined with an appropriate amount of the block copolymers to form a therapeutic composition solution. The therapeutic composition when administered to the site of aneurysm in a patient, forms a hydrogel in situ that remains at an aneurysm to provide sustained release of the tissue stabilization agent. The physico-chemical effect of the tissue stabilization agent on the resulting gel formulation are taken into consideration by investigating the effect of variables such as pH, gelation temperature, solubility, water content, and viscoelasticity. The hydrogel can be biodegradable. For these embodiments, the release profile of the biodegradable hydrogel is additionally affected by the biodegradation of the hydrogel itself. In some embodiments, the tissue stabilization agents are additionally embedded in polymers to form nanoparticles before forming a dispersion with the precursors of hydrogel.

One of the commercially available block copolymers for hydrogels are Pluronic™ polymers that generally comprise polyoxy-propylene/polyoxy-ethylene or polyoxy-ethylene/polyoxy-propylene/polyoxy-ethylene block copolymers. Hydrogels from the crosslinking of these block copolymers and similar compositions can be referred to as Pluronic™ hydrogels. The resultant hydrogel is additionally biodegradable. Poloxamer 407 hydrogels in particular are used as drug delivery vehicles for short term, as well as a combination of this hydrogel with other delivery vehicles e.g. PLGA nanoparticles to provide slow release profiles for extended period. Poloxamer 407 is a triblock copolymer consisting of a central hydrophobic block of polypropylene glycol flanked by two hydrophilic blocks of polyethylene glycol (PEG). The approximate lengths of the two PEG blocks are 101 repeat units while the approximate length of the propylene glycol block is 56 repeat units. Poloxamer 407 has an average molecular weight of 12.6 kDa and a melting point of 56° C. Poloxamer 407 is also known by the BASF trade name Pluronic™ F127 and commercially available from BASF. Gel forming polymers like poloxamer 407 are in situ gellable hydrogels and are of interest as delivery vehicles since they provide soft, penneable, and hydrophilic interfaces with body tissues. They are also listed in US, European pharmacopoeia and FDA's inactive ingredient database. Poloxamer 407 has been evaluated for its toxicity potential and is acceptable for use as a vehicle to achieve drug delivery. The block copolymers used for the gelation directly affect the gelation temperature and other significant properties of the final hydrogel, for example, the rate in which an active agent is release from the hydrogel.

Pluronic™ block copolymers when further modified can exhibit a variety of gelation properties to address different delivery needs. For example, Pluronic™ polymers can be coupled with an agent that has a functional group which can be further modified to introduce biologically active agents. The resultant final polymer can have improved thermal gelation temperature and affinity to cells such as those disclosed in WO 2007/064152A to Han et al., entitled “Injectable Thermosensitive Pluronic Hydrogels Coupled With Bioactive Materials for Tissue Regeneration and Preparation Methods Thereof,” incorporated herein by reference. Alternatively, Pluronic™ polymers can be combined with other polymers such as PLGA polymer building blocks to from thermosensitive, biodegradable hydrogels such as those disclosed in the published PCT applications WO 01/41735A to Shah et al., entitled “Thermosensitive Biodegradable Hydrogels Based on Low Molecular Weight Pluronics,” incorporated herein by reference. Block copolymers discussed here as well as other hydrogels precursors suitable for introduction into a patient can be similarly used.

Drug release rates from Pluronic™ hydrogels alone tend to be relatively rapid depending on the pore size, extent of cross-linking, and nature of the incorporated drug, and typically follow first order kinetics. Other hydrogel formulations for introduction into a patient are known in the art and can be adapted for use as a delivery vehicle as described herein. In general, the final concentration of the polymer in the final therapeutic composition can be in the range of about 5% to about 98% by weight, and the concentration of tissue stabilization agent in the therapeutic composition can be in the range of about 0.05 to about 100 mg/mL. Additionally, the hydrogel can be in the range of 5-95%, 7-80%, 8-75%, 9-70%, 10-60%, 12-50%, or 15-40% by weight, and the tissue stabilization agent can have concentration that is in the range of about 0.05-100 mg/mL, 0.1-95 mg/mL, 0.2-90 mg/mL, 0.5-80 mg/mL, 1.0-70mg/mL, 2.0-60mg/mL, 5-50 mg/mL, or 10-40 mg/mL in the hydrogel precursor solution. In some embodiment, the hydrogel used is Pluronic™ F-127 and in the range of about 20-40% by weight relative to the overall weight of the therapeutic composition. In some embodiments, the tissue stabilization agent is PGG that has a concentration in the range of about 0.1-50 mg/mL in the hydrogel precursor solution. In one embodiment, the concentration of the PGG is in the range of about 0.1-2 mg/mL. A person of ordinary skill in the art will recognize that additional ranges of concentrations within these explicit ranges are contemplated and are within the present disclosure.

Polymeric particles for drug delivery generally include, for example, biocompatible polymers and may or may not be spherical. The polymeric particles generally can have an average particle diameter of no more than about 5 microns, in further embodiments no more than a micron and in additional embodiments no more than about 250 nanometers, where the diameter is an average dimension through the particle center for non-spherical particles. The delivery of drugs using nanoparticles and microparticles is described further for example in published U.S. Patent application 2006/0034925 to Au et al, entitled “Tumor Targeting Drug-Loaded Particles,” incorporated herein by reference.

In general, it can be advantageous to form the nanoparticles from a bioresorbable polymer binder since the gradual dissolution of the polymer binder can facilitate release of the stabilization agent from the particles. Any suitable biocompatible bioresorbable polymer generally can be used. Suitable bioresorbable polymers include, for example, dextran, hydroxyethyl starch, gelatin, polyvinylpyrrolidone and combinations thereof. In further embodiments, suitable bioresorbable polymers comprise polyhydroxy acids and copolymers thereof, such as poly(caprolactone), poly(dimethyl glycolic acid) or poly(hydroxy butyrate) as well as polymers and copolymers of lactic acid and/or glycolic acid. The formation of nanoparticles from poly(lactic-co-glycolic acid (PLGA) is described in examples below. PLGA is a copolymer of polylactic acid (PLA) and polyglycolic acid (PGA). Depending on the ratio of lactide to glycolide used for the polymerization, different forms of PLGA can be obtained. Polymers comprises primarily of PLA or PGA only can also be used. As described further below, the use of a combination of the tissue stabilization agent embedded micro/nanoparticles within a hydrogel can provide a synergistic delivery advantage. Thus, improved delivery of aneurysm stabilizing compositions described herein can be more effectively delivered using the hydrogels and/or the particles described herein.

For prolonged tissue stabilization agent delivery, other controlled release delivery vehicle (such as nanoparticles) can be entrapped within hydrogels without any detrimental effects. The incorporation of nanoparticles, besides providing good control of the release of the encapsulated stabilization agent, can have additional advantages, such as isolation of the drug, slower release rates, improved residence times, and achievement of different release profiles. Although nanoparticles alone can be used to achieve long term drug release of weeks to months, such vehicles typically do not result in constant release profiles. Nanoparticles can exhibit an initial rapid burst release as a result of surface associated stabilization agent. Moreover, localization of nanoparticles to the site can be difficult.

Particles, such as nanoparticles, embedded within hydrogels are of special interest because the hydrogel matrix prevents stabilization agent degradation, allows local delivery, and also allows additional control over the release kinetics of the stabilization agent. Furthermore, the duration and levels of stabilization agent released from nanoparticles can be easily modulated by altering formulation parameters such as stabilization agent-to-polymer ratio, polymer molecular weight, and composition. The loading of nanoparticles within a hydrogel can be adjusted to achieve a desired amount of tissue stabilizing agent to the patient. In some embodiments, the nanoparticles comprise an elastin stabilization agent combined with the particle forming polymer. In some other embodiments, the nanoparticles comprise a collagen stabilization agent combined with the particle forming polymer. In yet some other embodiments, the nanoparticles comprise a combination of a collagen stabilization agent and an elastin stabilization agent. In some embodiments, the nanoparticles can be in the range of about 0.5-95, 1.0-90, 2.0-80, 2.5-70, 5-60, 7-50, 10-40 or 20-30 weight percent in the hydrogel. In one embodiment, the nanoparticles are in the range of about 2 to 60 weight percent of the overall therapeutic composition. A person of ordinary skill in the art will recognize that additional ranges of nanoparticle loading within a hydrogel-based therapeutic composition are contemplated and are within the present disclosure.

Specifically, for example, PGG-PLGA nanoparticles can be prepared by emulsion solvent evaporation technique which is disclosed in detail in prophetic example 1. Polymer composition, drug loading and particle size distribution are significant parameters to select based on clinical needs. The poly(lactide-co-glycolide) (PLGA) copolymers can consist of various ratios of lactic acid or lactide (LA) and glycolic acid or glycolide (GA). The copolymer can have different average chain lengths, resulting in different internal viscosities and differences in polymer properties. In some embodiments, the nanoparticles have an average size of about 0.1 nm to about 5 μm, about 1 nm to about 1 μm, about 10 nm to about 1 μm, about 50 nm to about 1 μm, about 100 nm to about 1 μm, about 250 nm to about 900 nm, or about 600 nm to about 800 nm. In some embodiments, the sizes of the nanoparticles have an average diameter in the range of 50-500 nm. In one embodiment, the nanoparticles have an average diameter of around 100-200 μm. In some embodiments, the tissue stabilization agent embedded in the nanoparticles can be in the range of about 0.05-99, 0.1-95, 0.5-90, 1.0-80, 2.5-70, 5-60, 7-50, 10-40 or 20-30 weight percent to the nanoparticle. In some embodiment, the tissue stabilization agent is in the range of about 0.05 to 50 weight percent to the nanoparticle. A person of ordinary skill in the art will recognize that additional ranges of concentrations within these explicit ranges are contemplated and are within the present disclosure.

In some embodiment, it may be advantageous to use tissue stabilization agent itself as delivery vehicle. For example, PGG formulations have been shown to form a gel under certain conditions. The conditions, such as concentration of PGG and pH during formation of the gel influence the resulting gel properties. In some embodiments, the PGG gel can be formulated to dissolve around 37° C., the body temperature of a patient. Additionally or alternatively, PGG can be formulated as a gel that remains its gel form at around 37° C. or higher temperatures. The gel form PGG can be used as drug delivery vehicle, for example, a slow release delivery vehicle for collagen stabilization agent, with properties adjusted as desired. Thus, the PGG would be both a delivery vehicle and a stabilization agent. The gel form of PGG can also be used in combination with other delivery vehicles such as hydrogel and/or poly(lactic-co-glycolic acid) (PLGA) nanoparticles to provide release profiles for short or extended period for a stabilization agent.

The use of PGG formulations for the delivery of polypeptide based treatment agents has been described for example in U.S. Pat. No. 7,323,169 to Goldenberg et al., entitled “Sustained Release Formulations,” incorporated herein by reference. PGG forms precipitates with agent of interest which is then isolated and dried to form a powder. The powder can be used as nanoparticles to be delivered to aneurysm for treatment. Epigallocatechin gallate (EGCG) can similarly be used as a delivery vehicle. These approaches can be adapted for the delivery of PGG or EGCG itself as well as collagen stabilization agent such as Glu. The particles can also be used in combination with other delivery vehicles such as hydrogel and/or nanoparticles with optional collagen stabilization agent encapsulated within the hydrogel and/or nanoparticles.

As described further in the examples below, local application of tissue stabilization agent such as PGG (applied as a solution using soaked gauze) was effective in suppression of AAA in rats. Different approaches for PGG delivery are developed in the discussion herein as well as related general approaches. Collagen stabilization agent such as glutaraldehyde (Glu) can likewise be incorporated alone or in combination with elastin stabilization agent such as PGG. For example, treatment of AAAs or other aneurysms can use: (1) hydrogels, such as Pluronic™ gel comprising a tissue stabilizing agent, such as PGG and/or Glu, (2) tissue stabilizing agent loaded polymeric nanoparticles: PGG alone, Glu alone or PGG+Glu, (3) hydro gel comprising polymeric nanoparticles of (2), (4) Pluronic™ gel comprising PGG and/or Glu and further comprising polymeric nanoparticles of (2) or the like to form therapeutic compositions with desired controlled release profile.

It is generally helpful to maintain the concentration of the stabilization agent within an effective window for a time period sufficient to achieve the desired effect with respect to more effective tissue stabilization and to avoid excessive concentrations, which may lead to side effects at the site of aneurysm with the delivery vehicle. The window of concentrations can be dependent on the particular tissue stabilization agent, and the appropriate concentrations can be evaluated based on the teaching herein along with empirical evaluations as outlined in the examples and prophetic examples below. Beside the general property of the hydrogel and/or nanoparticles associated with the delivery vehicle, the controlled release profile of the delivery vehicles can be additionally modulated by conditions such as pH, salt form, and concentration of the stabilization agent.

### Delivery Options for Therapeutic Composition

The therapeutic composition discussed herein can be applied to the aneurysm site in an intravascular procedure, a perivascular procedure, or a combination thereof. In some embodiments, the therapeutic composition can be applied to the outside of the aneurysm vessel, which would gel around the aneurysm vessel. The mechanical properties of the therapeutic composition upon gelling around the aneurysm vessel can be adjusted so the gelled therapeutic composition stays around the vessel and additionally anchor itself to the surrounding tissue. Non-invasively delivery method such as laparoscopy can be employed to deliver the composition.

Treatment with a tissue stabilizing agent can be combined with mechanical stabilization. In particular, a perivascular girdle wrap can be placed over the exterior of the aneurysm to provide mechanical stabilization along with the chemical stabilization, such as the one shown in FIG. 2C. For example, the therapeutic compositions can be coated along the interior of the wrap and/or embedded in the material of the wrap. The wrap provides a close contact to the aneurysm site for consistent drug release in addition to the delivery vehicle described herein. In these embodiments, the girdle wrap physically strengthens the vasculature at the aneurysm site to prevent it from bursting. The stabilization agents act to stabilize and strengthen the tissue of the vessel along with inhibiting further degradation of the vessel at the location. The delivery vehicle modulates the release rate of the tissue stabilizing agent within the therapeutic composition. The wrap can be formed from biocompatible polymers, such as polyesters, that can be formed into woven or non-woven fabrics. Alternatively, the wrap can be formed from bioresorbable material such as those disclosed in U.S. Pat. No. 6,258,122 to Tweden et al. entitled “Bioresorbable annuloplasty prosthesis”, incorporate herein by reference.

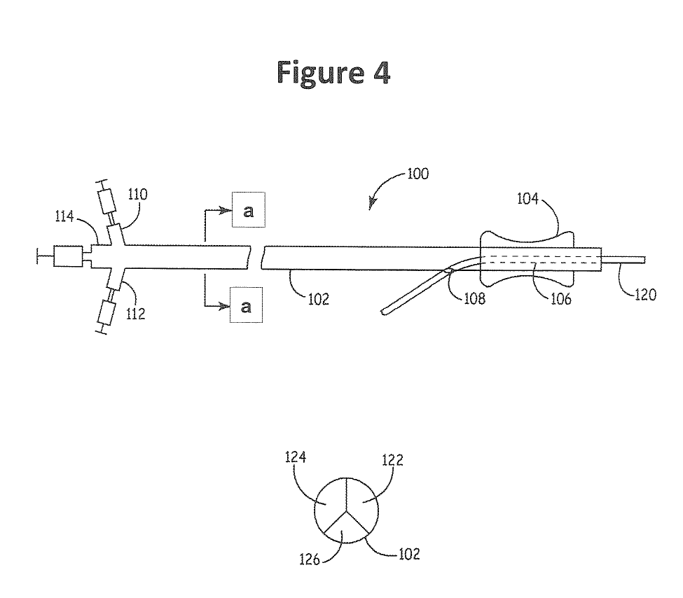

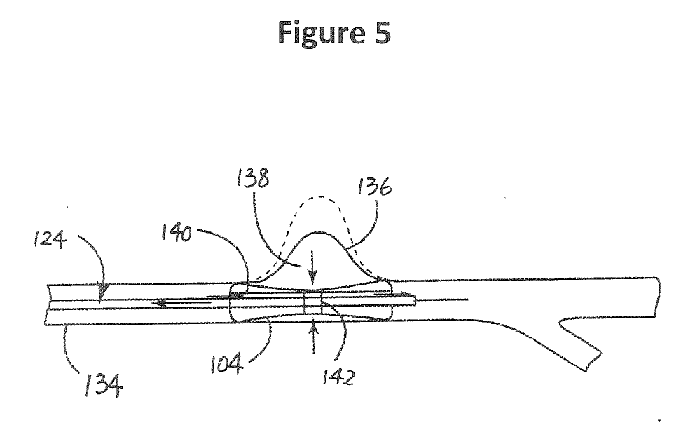

In some embodiments, the therapeutic composition can be applied to the aneurysm site in an intravascular approach if the site can be isolated from the blood flow temporarily. Delivery devices that delivers the therapeutic composition to an isolated volume at the aneurysm are described for example in U.S. patent application Ser. No. 12/173,726 (the '726 Application) to Ogle et al, entitled “Devices for the Treatment of Vascular Aneurysm,” incorporate herein by reference. The delivery devices offer the possibility of isolating the aneurysm for treatment with the stabilization agents while allowing the regular blood flow to by-pass the site of aneurysm. The aneurysm is normally aspirated first with the delivery device to alleviate pressure and followed by the delivery of a therapeutic composition containing the tissue stabilization agents. The delivery devices have a variety of embodiments to suite different application needs. The devices optionally have an aspiration device to improve the effectiveness of the treatment based on the ability to relieve the pressure at the aneurysm as well as having the ability to remove compositions in the vicinity of the aneurysm. The devices shown in FIGS. 4 and 5 illustrate the general concept disclosed in the '726 Application. Additional embodiments of the device are illustrated in the '726 Application. In some embodiments, intravascular treatment using the devices disclosed in the '726 Application can be combined with the perivascular treatment such as using laparoscopic procedure to deliver the therapeutic composition outside the aneurysm or using the perivascular girdle described above.

A rapid exchange delivery device is shown schematically in FIG. 4 (Top). Isolation/delivery device 100 comprises a shaft 102, a sealing element 104, a guide lumen 106 with a guide port 108, and three access ports 110, 112, 114 that provide for delivery or removal of fluids through three corresponding lumens. A guidewire 120 is shown extending through a separate guide lumen 106, which is attached to the shaft. FIG. 4 (Bottom) shows a cross section of shaft 102, which comprises three flow lumens 122, 124, 126 that, respectively, are in fluid communication with access ports 110, 112, 114.

When placed inside a vessel 134 to isolate an aneurysm 136 as shown in FIG. 5, the sealing element 104 of device 100 is transformed into an extended configuration forming an isolated volume 138 inside the vessel 134. The transition to the extended configuration can be performed based on the particular design of the device. For example, the transition to the extended configuration can be preformed, for example, through the filling of one or more balloons, through the release of a self extending member from a sheath or through the use of an actuation element. Flow in the vessel is maintained through a by-pass channel 140 of the sealing element 104. A fluid exchange portion 142 is configured for the exchange of fluids between a lumen such as 124 of device 100 and isolated volume 138. In an optional step, blood is withdrawn from isolated volume 138 through the fluid exchange portion 142 and lumen 124 in device 100. The withdrawal of blood decreases the pressure in isolated volume 138, which can result in decrease or elimination of the distortion of the vessel at the aneurysm 136.

The access ports 110, 112, 114 of the device 100 can be connected to flow devices such as syringes, pumps, or the like, or combinations thereof. For example, an empty syringe can be connected to port 110 to withdraw fluid from the isolated volume 138 to reduce pressure at the site of aneurysm 136. Another syringe loaded with the therapeutic composition disclosed herein can be connected to port 112 to deliver the therapeutic composition discussed herein to the isolated volume 138 at aneurysm 136 inside the vessel 134. Luer fitting and other appropriate fittings, such as those known in the art, can be used to attach the flow devices to the access ports.

In some embodiments, a hydrogel can be selected to gel upon application to the patient after being delivered to the site of aneurysm using the delivery/isolation device discussed above. The gelling process holds the compositions in association with the aneurysmal tissue. Upon proper setting of the material, the delivery/isolation device can be removed. Similarly, nanoparticles embedded with tissue stabilizing agent can be applied as a dispersion using the delivery/isolation device. The nanoparticles in the dispersion can penetrate into the aneurysmal tissue to provide its effect. Alternatively, the nanoparticles can be delivered with a hydrogel, with the hydrogel maintaining the nanoparticles in the vicinity of the aneurysmal tissue.

In some embodiments, using the delivery device described herein, an effective amount of collagen stabilization agent, such as glutaraldehyde, is delivered to the isolated aneurysm tissue after the initial fluid aspiration to relieve pressure. The collagen stabilization agent is allowed to interact with the aneurysm tissue for a period of time before being aspirated out. The time period can be for example, about 5, 10, 15, 20, 25, or 30 mins, and can be longer in some embodiments. Optionally, the collagen stabilization agent treated aneurysm tissue can be rinse with a buffer such as saline before further treatment using the therapeutic composition described herein. Because the delivery device can have multiple ports connected to multiple flow devices, the delivery device can be maintained in the vessel while the content of the flow devices is switched. After the initial treatment with collagen stabilization agent, the elastin stabilization agent such as PGG can be delivered for example with block copolymer described herein to aneurysm. Once reaching the aneurysm tissue, the block copolymers forms hydrogel in situ, locking the PGG inside the hydrogel for sustained release. The hydrogel optionally can have nanoparticles encapsulating PGG for longer release. Alternatively, nanoparticles encapsulating PGG without hydrogel can be administered as a dispersion. The solution in the dispersion can optionally have PGG and or glutaraldehyde.

The collagen stabilization agent treatment step and the elastin stabilization agent treatment step can be performed sequentially without withdrawal of the delivery device or can be performed as separate steps with withdrawal of the delivery device in between. Based on the condition of the aneurysm, the treatments steps can be preformed multiple times with different combination of therapeutic compositions and time intervals. Sometimes the treatment steps can be repeated periodically or when the sustained release of the tissue stabilization agent is significantly diminished. Diagnostic method such as using the diagnostic biomarkers disclosed in the '384 application can be used to help determine the dose and duration of treatment.

### Shipping and Storage Options for Therapeutic Composition

The tissue stabilization agent can be shipped and stored under a variety of conditions in combination with the delivery vehicle. For example, in addition to the delivery vehicle, the stabilization agent can additionally comprise pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. Excipients include pharmaceutically acceptable stabilizers and disintegrants.

The compositions or their components are generally stored in sterile containers that are suitable for distribution. The containers are generally marked with expiration dates based on the safe shelf storage time. The containers are generally also shipped with appropriate FDA approved instructions and warnings. In some embodiments, the tissue stabilization agent and the delivery vehicle are stored separately until right before being administered into a patient. In some other embodiments, the tissue stabilization agent are mixed with the delivery vehicle to form the therapeutic composition and stored accordingly. In yet some other embodiments, a portion of the tissue stabilization agent can be combined with the delivery vehicle to form a mixture while the other portion of the tissue stabilization agent is not combined with the mixture to form the final therapeutic composition until right before being administered into a patient. In some embodiments, the therapeutic composition can be packaged and distributed in the lumen of a syringe. In some embodiments, the various components or forms of the therapeutic composition can be package in the lumen of different syringes.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. All patents, patent applications, and publications referenced herein are hereby incorporated by reference herein to the extent that the incorporated material is not contrary to any of the explicit disclosure herein.

### EXAMPLES

Phenolic tannins such as PGG bind to the elastin component of aorta and increase the resistance of arterial tissue to degradation by elastase. This resistance to elastase was effective even when PGG was applied to tissues which had already experienced some level of enzymatic degradation. In a series of in vivo pilot studies presented in Examples 5 and 6, it is shown that perivascular application of PGG solution limits formation and progression of abdominal aortic aneurysms in a rat model. Not to be bound by any particular theory, the binding of PGG to arterial elastin is believed to protect elastin from enzymatic degradation and thus limits aneurysm progression. Additionally, collagen stabilization agent such as Glu alone or in combination with PGG has shown additional protection to aortic tissue.

### Example 1

### Tannins Bind to Pure Aortic Elastin

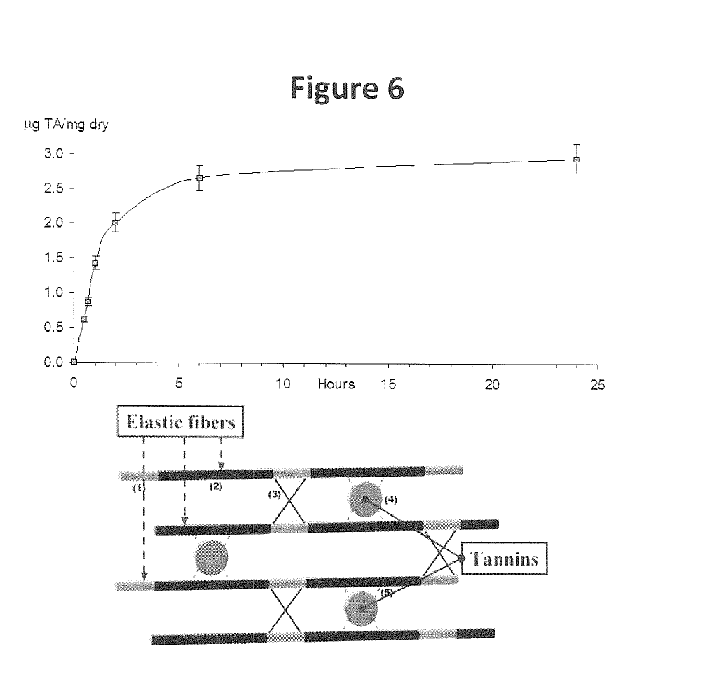

In order to determine kinetics of tannin binding to elastin, pure elastin strips obtained from porcine aorta were incubated with tannic acid (TA) for up to 24 hours. The concentration of TA in solution was assayed with the Folin-Denis method. The diagram at the top of FIG. 6 shows TA binding values normalized to dry weight of pure elastin strips. The kinetics shows a rapid uptake of TA, clearly indicating tannin binding to elastin. All data points between 0-6 hrs are statistically different with p<0.05. The bottom diagram of FIG. 6 is a representative schematic diagram of interactions between TA and elastin. The hydrophobic domains (2, black segments) are areas in the elastin molecule that are susceptible to elastase cleavage. TA and PGG molecules (4, round structures), with an affinity for these hydrophobic areas, likely bind to these regions within elastin molecules, and establish multiple hydrogen bonds (5, dashed lines) between their hydroxyl moieties and regions of neighboring elastin molecules, resulting in improved elastin stabilization. Desmosine crosslinks (3, X) within the hydrophilic regions (1) of elastin molecules.

### Example 2

### Tannins Protect Pure Aortic Elastin from Degradation

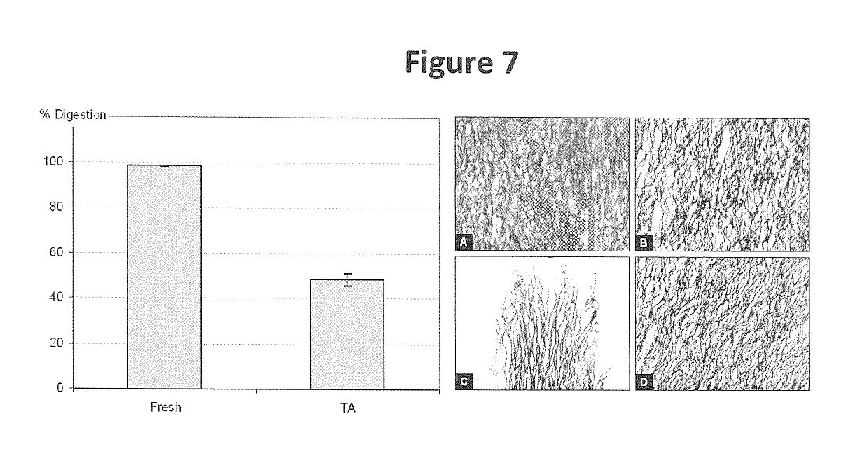

To investigate the potential of tannins as elastin stabilizing reagents, pure elastin obtained from porcine aorta was treated with tannic acid (TA) and the resistance of the treated porcine aorta to elastase was tested. Porcine aorta was chosen because large quantities of fresh tissue were easily obtainable. FIG. 7 shows tannic acid mediated stabilization of pure elastin against the action of elastase. Histology of fresh porcine aorta is shown in FIG. 7A. Purified elastin from porcine aorta was obtained using sodium hydroxide treatment followed by collagenase digestion. The smooth muscle cells, collagen and ground matrix are absent from purified aortic elastin. This resulted in an intact, highly purified elastin shown in FIG. 7B which contains a minimal degree of random peptide cleavage, low hexosamine levels, and undetectable protein impurities. Elastase completely digested pure elastin strips, while addition of a 0.3% TA pretreatment step increased stability towards enzymatic digestion by almost 50%, as evaluated by mass loss shown in the graph at the left of FIG. 7 (p<0.05, n=6). Gravimetric data were further confirmed by histology and shown in FIG. 7A-D. Since histological analysis of elastase-digested elastin control was not possible at 48 hours due to complete (˜100%) degradation, we exposed pure elastin to elastase for only 1.5 hours, an interval which yields about 50% digestion and mass loss. Partially degraded elastin exhibited extensive fraying and massive loss of fibers as shown in FIG. 7C. However, elastase treatment of TA-stabilized elastin showed that the aortic structure had been remarkably preserved, without massive loss of elastin integrity for up to 48 hours as shown in FIG. 7D.

### Example 3

### PGG and TA Protect Fresh Aorta from Enzymatic Degradation in Vitro

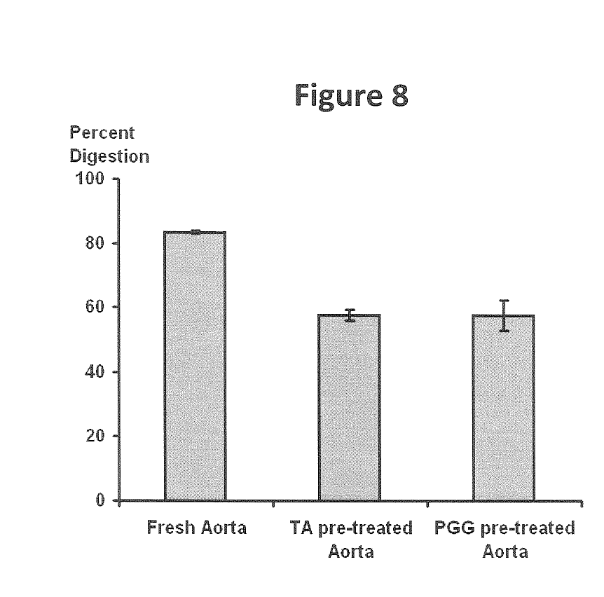

Resistance to elastase digestion was tested using fresh, untreated aorta and aorta treated with 0.3% TA or 0.15% PGG (equimolar concentrations). Treatment with TA or PGG dramatically increased resistance of aorta to elastase as shown in FIG. 8, yielding digestion values that were significantly lower than those of control, untreated fresh aorta (p<0.05). The differences between digestive values in TA and PGG samples were not significant (p>0.05). This is an accelerated digestion study, where high concentrations of enzyme were used. Such high enzyme activities are not expected to occur in vivo.

### Example 4

### PGG Binds to and Protects Partially Degraded Elastin

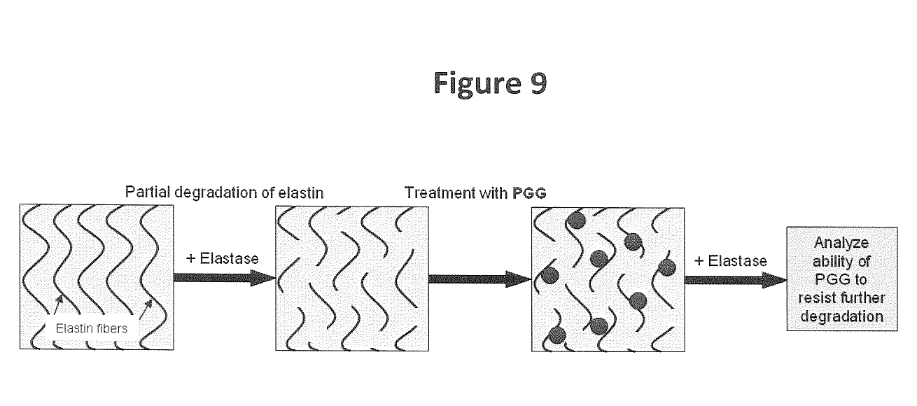

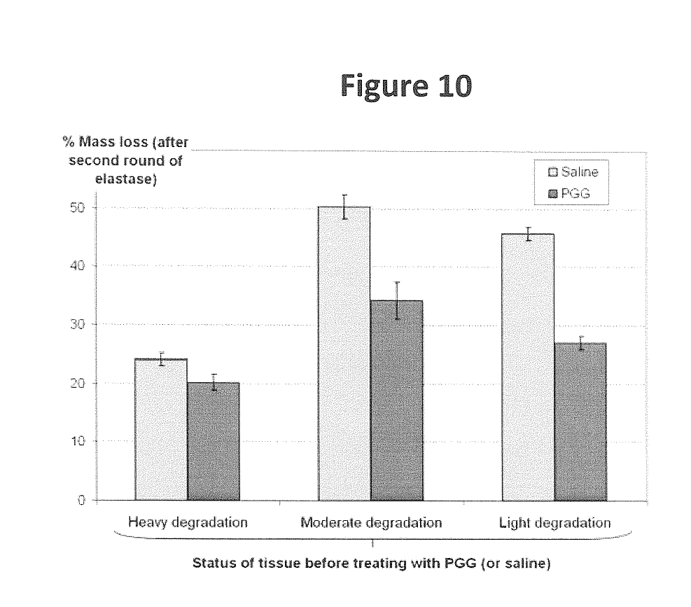

In clinical setting, PGG would be applied to diseased tissues which would have likely already experienced some level of tissue degradation. As a result, it is worthwhile to evaluate the efficacy of PGG on arterial specimens which possessed varying quantities and qualities of elastin. These varying levels of elastin can be simulated by individually subjecting tissues to a range of elastin-degrading enzyme concentrations as shown in FIG. 9 to imitate the degradation found in the different stages of aneurysmal development, such as early-stage, moderate, and late-stage aneurysms. Samples of porcine aorta were subjected to one of the following concentrations of elastase for 24 hrs: 20 U/mL, 1 U/mL, 0.5 U/mL, or 0 U/mL (buffer control). Following the first round of digestion, samples were treated with 0.1% PGG (or saline as control) for just 30 minutes at 37° C. Once treated, samples were exposed to a second round of elastase (5 U/mL, 48 hrs) to evaluate the effectiveness of the PGG treatment to resist any further degradation. Dry weights after the first round of elastase were compared to dry weights after the second round of elastase in order to calculate percent mass loss. As shown in FIG. 10, in comparison to saline controls, PGG is most effective on the tissues that had been lightly or moderately degraded with 0.5 U/mL and 1 U/mL elastase, simulating early-stage or moderate aneurysms. However, it is important to note that even those PGG-treated samples which were initially heavily degraded with 20 U/mL elastase also showed some improvement in resisting further elastolytic degradation when compared to saline-treated controls (p<0.05) in FIG. 10.

### Example 5

### Periadventitial PGG Treatment Reduces Abdominal Aneurysm Formation

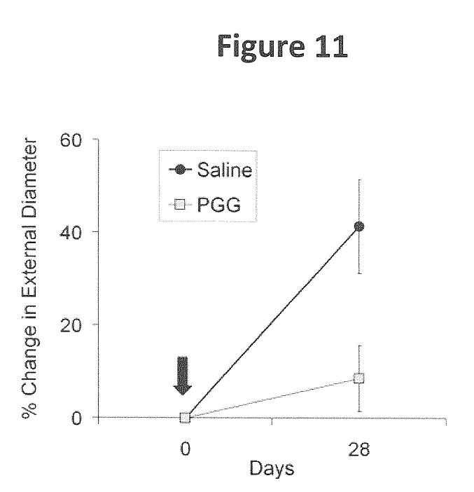

Pelivascular application of calcium chloride (CaCl₂) to the infrarenal abdominal aorta of rodents is an accepted rat aneurysm model. It involves exposure of the abdominal aorta through a midline incision, using gauze to apply CaCl₂ solution directly onto the aorta for 15 minutes, followed by surgical closure. Using this aneurysm model, we evaluated the effect of a single PGG application on development of abdominal aortic aneurysms in rats. For this study, we exposed the abdominal aorta in adult rats (n=12), measured the aorta diameter using digital photography and applied a 0.03% PGG solution in saline for 15 minutes using soaked gauze. After rinsing in saline, we induced aortic injury with the 15-minute application of 0.5 mol/L CaCl₂ solution. Control rats (n=12) were treated with saline for 15 minutes, rinsed and then treated with 0.5 mol/L CaCl₂ solution for 15 minutes. After 28 days, we re-exposed the abdominal aorta under general anesthesia, measured the external diameter, then euthanized the rats and collected the aorta for analysis. Comparative measurements of the external aortic diameter of control (saline-treated) rats at day zero and 28 days after surgery (1.395 +0.052 mm and 1.939±0.112 mm, respectively) revealed a mean increase in diameter of 42±10% (p<0.05). By comparison, aortas that were exposed to PGG exhibited minimal (8±7%) increase in diameter after 28 days from 1.564±0.064 mm to 1.676±0.097 mm as shown in FIG. 11.

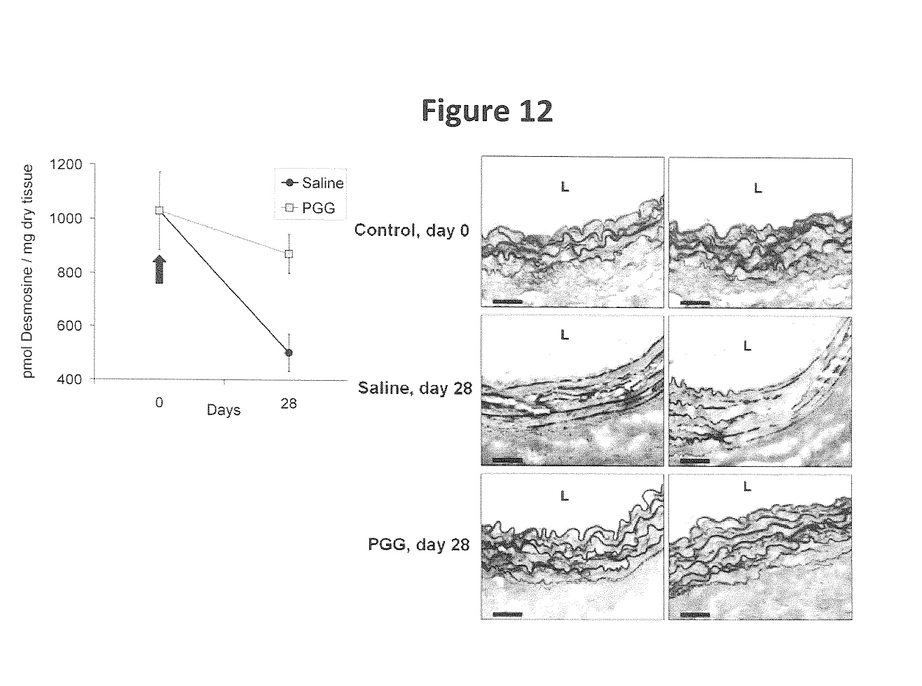

Along with aortic dilatation, perivascular application of CaCl₂ induced major changes in vascular elastin content and integrity as shown by analysis of desmosine, an amino acid which is specific to elastin, as well as histology, all shown in FIG. 12. As compared to non-surgery control aorta collected from age-matched rats (represented as day 0), aortic elastin content in the saline-treated control group diminished by almost 50%, as suggested by the drastic drop in desmosine content in the left graph of FIG. 12. Histology shown by using Verhoeff van Giesson stain (VVG) on this same group exhibited characteristic flattening and fragmentation of the elastic laminae at 28 days after injury, shown in right middle panel of FIG. 12.

Conversely, aortas from the PGG group exhibited little decrease in elastin content as compared to normal non-surgery control aorta (less than 15% loss of desmosine, p>0.05 versus non-surgery control) and excellent preservation of elastic laminae integrity and waviness, suggesting that PGG delivery effectively prevented elastin degeneration in this animal model. In addition, quantitative PGG content analysis of explanted aorta revealed that rat aortas explanted 28 days after PGG application contained slightly lower (data not shown) but not statistically different amounts of PGG in comparison to rat aortas explanted at day 0 immediately after PGG application: 1.2±0.4 μg PGG/mg dry tissue vs. 1.8±0.6 μg PGG/mg dry tissue; p>0.05. These data indicate that in vivo binding of PGG to aortic tissue is relatively stable for a minimum of 28 days in this accelerated model.

### Example 6

### Periadventitial Treatment of Aneurysmal Aorta with PGG Limits Aneurysm Progression in Rodents

Using the same perivascular CaCl₂ aortic injury model described earlier, the ability of PGG to halt the progression of growing aneurysms was evaluated. By applying PGG to healthy abdominal aorta immediately before the CaCl₂-based aneurysm induction, the previously mentioned in vivo experiment essentially evaluated the effect of PGG on aneurysm formation. However, in order to create a more clinically relevant scenario, PGG was also applied to abdominal aortas of rats which were aneurysmal to investigate PGG's ability to hinder or halt aneurysm progression. In order to do this, rat aortas were treated with calcium chloride, the animals were closed, and AAA was allowed to develop for 28 days.

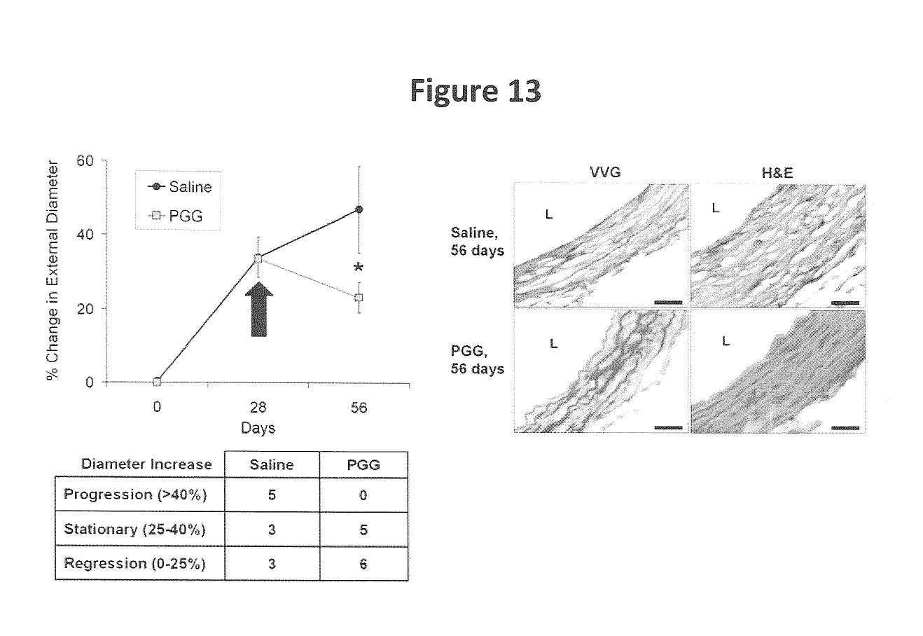

At this time point, the aneurysmal aortas were exposed by a second surgery, and PGG was applied perivascularly using gauze. As a control, saline was applied by the same means to the remaining aneurysmal aortas. AAA progression was followed for another 28 days in both groups. A progressive diameter increase, reaching a mean 47.1±11% increase at 56 days, was measured in the control group (n=11) and the results were illustrated in FIG. 13. Approximately half of the aneurysmal aortas significantly increased in diameter from day 28 to 56, indicating chronic AAA progression in this animal model.

Conversely, aneurysmal aortas that were exposed to PGG exhibited no increase in mean diameter at 56 days compared with day 28 mean values (n=11) as shown in FIG. 13. It is especially noteworthy that 100% of aortas in the PGG group (11 of 11) maintained the same diameter or exhibited a decrease in aortic diameter at 56 days compared with 28 days, shown in the table at the lower half of FIG. 13. The mean diameter value at 56 days for the PGG group was actually slightly lower than that at 28 days but not statistically significant (p>0.05).

At 56 days after injury, aneurysmal aortas exhibited extensive flattening, fragmentation, and degeneration of the elastic laminae in the control group. Overall tissue architecture was indicative of severe tissue degeneration as outlined by numerous gaps or lacunae, bestowing the aneurysmal aorta with a porous, “spongy” aspect. In contrast, PGG-treated aortas exhibited improved preservation of elastic laminar integrity and waviness and overall preserved tissue architecture as shown in FIG. 13. Overall, these results indicate that PGG application to aneurysmal aortas effectively hindered arterial dilatation and limited further degradation in this experimental model.

### Example 7

### Tissue Resistance to Elastase Degradation after Treatment

Tissue resistance to elastase degradation after treatment with various reagents is studied. Specifically, porcine carotid arteries were treated with saline (control, for 20 minutes), Glutaraldehyde (Glut) (for 20 minutes), PGG (for 20 minutes), or a combination of the two (Glut+PGG for 20 minutes, or Glut for 10 minutes followed by a separate incubation with PGG for 10 minutes). Concentrations of the reagents used were 0.6% (w/v) for Glut, 0.15% (w/v) for PGG and 9 g/L for physiological saline.

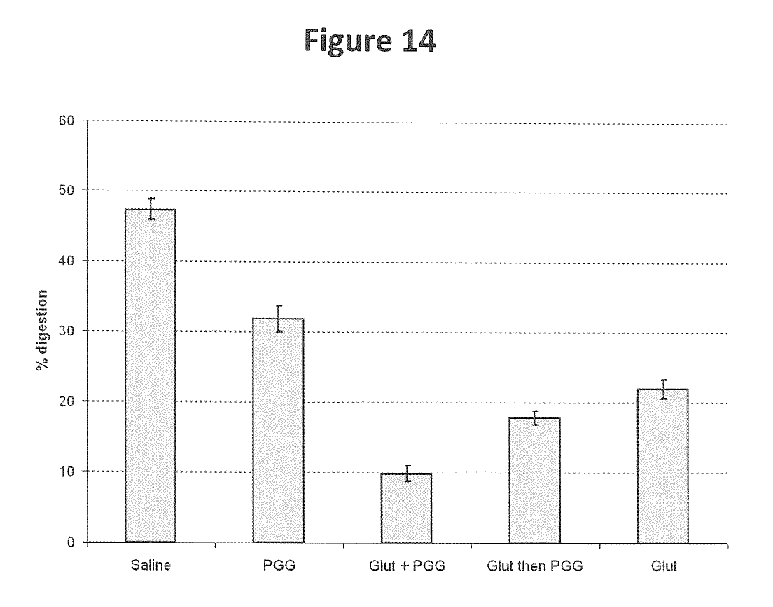

The treated tissue was then exposed to an in vitro elastase digestion assay to subject the treated tissue to digestion for 24 hrs. All experiments were conducted at 37° C. The percentage digestion of the arteries was measured after the assay and results are shown in FIG. 14. Because the values shown are percentage of digestion, the lower the value, the better the reagent used preformed in resisting elastase degradation. Individually, Glut and PGG each slightly improved the resistance of the tissue to degradation as compared to saline controls. When Glut and PGG are used together, either as a cocktail or sequentially as indicated above, there appeared to be a synergistic effect between the two reagents, resulting in very little degradation of the tissue. It should be noted that the digestion model used in this experiment is a very accelerated and aggressive digestion model.

### Example 8

### Mechanical Testing to Determine the Stiffness of the Treated Tissue

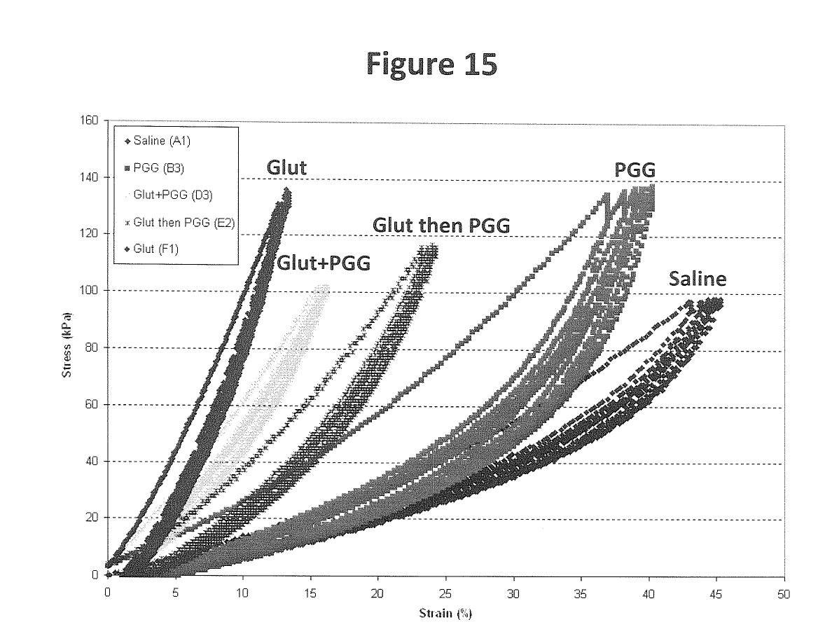

Porcine carotid arteries were treated using the conditions specified in Example 7. The treated tissues were then subjected to uni-axial tensile testing and the results are shown in FIG. 15. The degree of tissue stiffness is indicated by the slope of the curves. The more vertical curve corresponds to more stiffness. The more horizontal curve corresponds to less stiffness. As shown in FIG. 15, the saline treated control tissue is least stiff since it is essentially fresh native tissue. Glut treatment yielded the stiffest tissue. The inclusion of PGG in the treatment process made the tissue slightly less stiff. The stiffness of the resultant tissue can be tuned by using different ratio concentration combination of Glut and PGG.

### Example 9

### Opening Angle Test to Determine Mechanical Property of the Treated Tissue

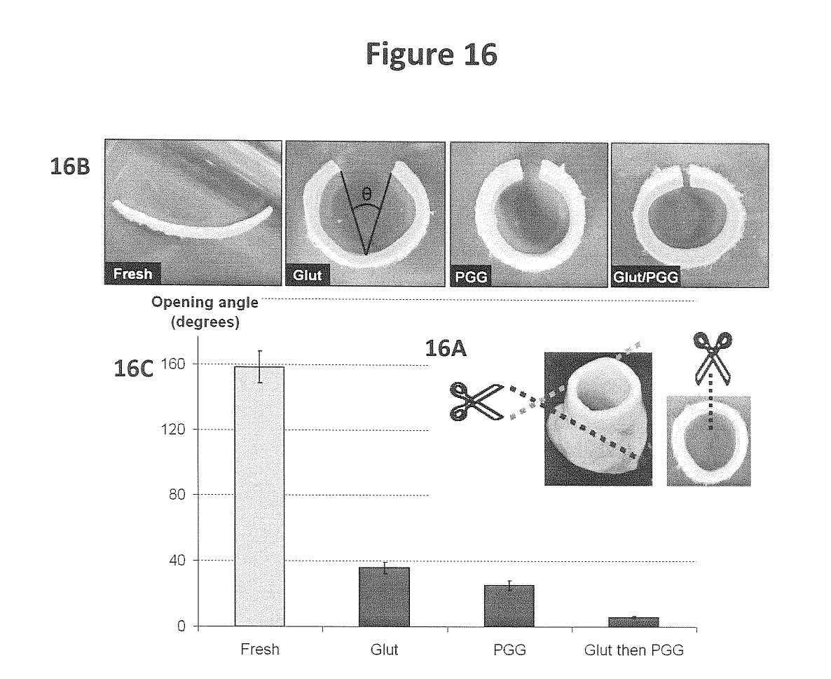

Porcine aorta was cut transversely into ring segments approximately 1 cm in height as shown in FIG. 16A. The rings were left untreated (fresh sample) or treated with Glut, PGG, or Glut then PGG (Glut/PGG). Glut treatment was performed with 0.6% (w/v) Glut for 1 day, and then 0.2% (w/v) Glut for 7 days, all done at room temperature; PGG treatment was performed with 0.15% (w/v) PGG for 4 days at 37° C. Glut/PGG treatment was performed with 0.6% (w/v) Glut for 1 day, and then 0.2% (w/v) Glut for 7 days at room temperature followed by 0.15% (w/v) PGG for 4 days at 37° C.

After treatments were completed, the aortic rings were immersed in water with the cross section of the aorta facing upward, allowing free movement of the aortic tissue. The aortic rings were cut once in the radial direction, as shown in FIG. 1 6A and allowed to “relax” and open for 15 minutes under water, and then digitally photographed. The photographs were shown in FIG. 1 6B. The digital photographs were then used to calculate the opening angle of each aortic ring graphically using Adobe Photoshop 7.0. The opening angle of each aortic ring was compared in graphical format in FIG. 16C. As shown in FIG. 16C, while the fresh sample has an opening angle of close to 160 degrees, the treatment with Glut has significantly altered the mechanical property of the aortic ring as shown by the significant reduction of the opening angle to close to 40 degrees. Treatment with PGG alone reduced the opening angle even further. The most significant reduction is seen in the treatment by Glut then PGG with an opening angle of less than 10 degrees.

### Example 10

### Tissue Resistance to Collagenase Degradation after Treatment

Tissue resistance to collagenase degradation after treatment with various reagents is discussed. Specifically, samples of porcine aortic wall were either left untreated (fresh) or treated with Glut alone or Glut followed by tannic acid (TA). Glut treatment was performed with 0.6% (w/v) Glut for 1 day, and then 0.2% (w/v) Glut for 7 days at room temperature; Glut/TA treatment was performed with 0.6% (w/v) Glut for 1 day, and then 0.2% (w/v) Glut for 7 days at room temperature followed by 0.15% (w/v) TA for 4 days at 37° C. The treated samples were rinsed 3 times (1 hour each) in 100 mL water, and lyophilized to record dry weight. Samples with the amount of 15 to 25 mg dry weight were immersed in 1.2 mL of type I collagenase (150 U/mL) dissolved in 50 mM Tris buffer, 10 mM CaCl₂, pH 7.4, and incubated at 37° C. with orbital shaking at 650 rpm for 24 hours. Following this exposure to collagenase, samples were centrifuged (10000 rpm, 10 minutes, 4° C.), individually rinsed three times in 1 mL water, lyophilized to obtain dry weight after collagenase, and the percent of digested tissue was calculated.

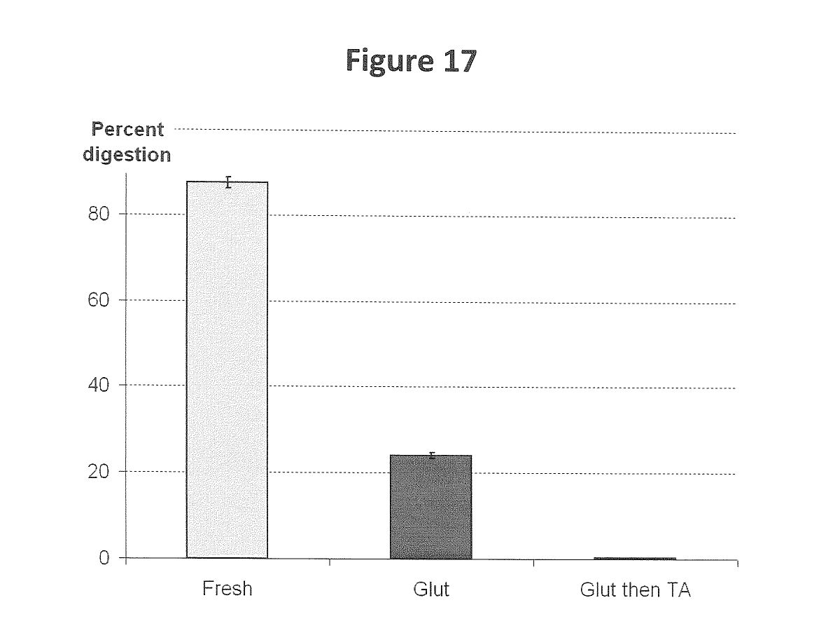

The percentage of tissue digestion was compared in graphical format in FIG. 17. As shown in FIG. 17, while over 85% of the fiesh sample has been digested, the percentage of the sample been digested has been reduced to slightly over 20% after treatment with Glut. Mass loss value for aorta treated with Glut and TA were essentially zero, suggesting that tannins may even enhance the ability of Glut to protect collagen from enzymatic degradation.

### Example 11

### Thermal Denaturation Temperature of Treated Tissues

The thermal denaturation temperatures (T_{d}), common indicators of collagen crosslinking density, were measured in samples from treatment groups using a differential scanning calorimeter (DSC) (Perkin-Elmer DSC 7; Boston, Mass.). The samples were treated under the conditions outlined in Example 10. The treated aortic wall samples (approximately 2 mm×2 nm) were sealed in aluminum pans, heated at a rate of 10° C. per minute from 20° C. to 110° C. T_{d} was determined as the temperature measured at the endothermic peak. This observed endothermic peak occurs at the temperature where collagen fibers unravel or denature, resulting in a measurable release of energy. Therefore, a higher denaturation temperature correlates into improved collagen crosslinking. The T_{d} data from the samples are recorded in Table 1. According to the data in Table 1, fresh untreated sample has T_{d} that is significantly lower than the Glut treated sample, indicating significant increase of degree of collagen crosslinking. The additional treatment with TA following the Glut treatment didn't result in significant increase in T_{d}.

TABLE 1

| | Thermal Denaturation |
|---|---|
| Treatment Group | Temperature (T_{d}) |
| Fresh | 68.37 ± 0.67° C. |
| Glut | 90.43 ± 0.27° C. |
| Glut then TA | 92.92 ± 0.93° C. |

### Prophetic Examples

### Evaluation of Release Kinetics of PGG-loaded Polymers

The following prophetic examples are intended to characterize PGG-loaded polymers and determine release kinetics of these short (Pluronic™) and sustained (nanoparticles dispersed in Pluronic™) release vehicles. In vivo studies are designed to confirm that PGG is appropriately delivered/released by these vehicles. The treatment described herein locally hinder elastin degradation, a hallmark of AAAs. Phenolic tannins such as tannic acid and penta-galloyl glucose (PGG) bind to elastin and thus increase its resistance to pancreatic elastase. In vivo results also indicate that PGG was effective in suppressing aneurysm formation and progression. PGG was delivered by simple placement of soaked gauze in these studies. Ideally, however, PGG could be locally delivered with minimally invasive surgeries.

In this example, PGG is delivered to the aneurysm site perivascularly, or through laparoscopic application. Two polymers, Poloxamer 407 (Pluronic™gel) and poly(lactic-co-glycolic acid) (PLGA) used in FDA approved formulations to deliver pharmacological agents are chosen as delivery polymers. These polymers are used to deliver PGG in a quick bolus-like dosage (Pluromic™ gel) or via prolonged release (Pluronic™ gel+PLGA nanoparticles). The release kinetics of short (Pluronic™) and sustained (nanoparticles dispersed in Pluronic™) release vehicles of PGG-loaded polymers were determined to locally deliver the required dosage of PGG to be effective against the growth/expansion of AAAs.

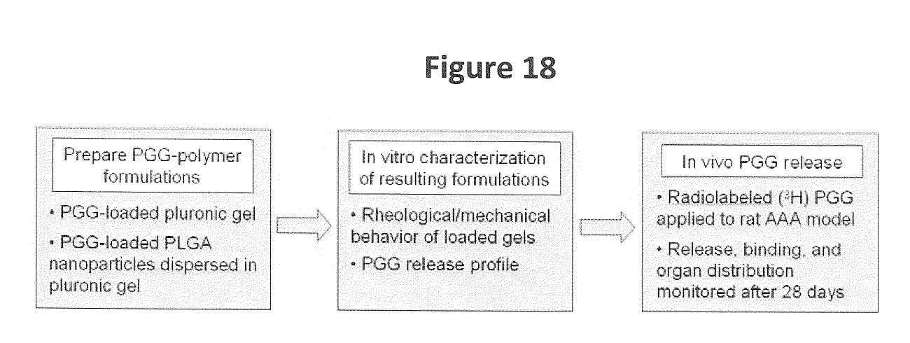

PGG is incorporated in the Pluronic™ and/or PLGA nanoparticle formulations. The release profile, polymer gelation, and mechanical properties in vitro of the formulations are optimized. The two optimized release formulations that deliver PGG for short (Pluromic™ hydrogel only) and prolonged release (PLGA nanoparticles dispersed in Pluronic™ hydrogel) were tested in vivo. Radiolabeled PGG is administered within a rat AAA model and evaluated 28 days later to determine release of PGG from the polymer formulations, as well as binding and organ distribution in vivo (FIG. 18).

### Prophetic Example 1

### Formulation of PGG Loaded Delivery Vehicles

Preparation of in situ Thermo Reversible Formulation of PGG-Pluronic™ Gels

The poloxamer gel is prepared by cold method. This method facilitates poloxamer dissolution and limits possible alteration. An appropriate amount of Pluronic™ F-127 (20-30% w/w) is added to cold sterile distilled water (˜4° C.), followed by additions of PGG (100 μg to be loaded for each application) and isotonic sodium chloride (9 g/L), and ultimately adjusted to pH 7.4. The formulation is stored at 4° C. to maintain complete dissolution, until gelling is to be performed at 37° C. The physico-chemical effect of PGG on the resulting gel formulation is evaluated by investigating pH, gelation temperature, solubility, water content, and visco elasticity.

### Preparation of PGG-Loaded Nanoparticles

PGG-PLGA nanoparticles are prepared by emulsion solvent evaporation technique. Briefly, an aqueous solution of PGG is emulsified into PLGA (varying copolymer ratio) solution in methylene chloride using a probe sonicator. The water in oil emulsion is further emulsified into an aqueous solution of polyvinyl alcohol (PVA) by sonication to obtain water in oil in water emulsion (w/o/w). The conditions for emulsification and the formulation composition are optimized to obtain nanoparticles. The multiple emulsion is stirred for approximately 24 hours followed by 1 hour in a desiccator under vacuum to remove any residual methylene chloride. Nanoparticles are recovered by ultracentrifugation at 25,000 rev/min. The nanoparticles are washed in distilled water to remove PVA and unentrapped PGG, then lyophilized for 48 hours to obtain dry powder. Encapsulation efficiency, drug loading, percentage yield, particle size distribution (particle size analyzer), surface morphology (scanning electron microscopy) and zeta potential are performed.

### Loading PGG Nanoparticles in Phuronic™ Gel

Pluronic™ solutions are prepared and chilled in the same manner as stated above. PGG loaded PLGA nanoparticles dispersed in different volumes of water is added in the Pluronic™ solution without using any co-solvents. After thorough stirring, 200 μl of solution is kept for gelling at 37° C. and their gelling time is recorded.

### Prophetic Example 2

### In Vitro Characterization of Gels and Nanoparticle Delivery Vehicles

### Rheological Behavior

Rheological behavior represents a significant part in the formulation of Pluronic™ gel preparations. The viscosity is considered as a quality control method in order to assess the behavior of the gels at body temperature. This includes flow curve studies (shear stress versus shear rate) to determine Newtonian and non Newtonian behavior of gels and the effect of temperature on sol-gel transition. Oscillatory studies using creep viscometer gives information on time-dependent changes of the viscoelastic properties, kinetics of gelation, and gelation time.

Characterization of Pluronic™ gels

In vitro determination of gel strength provides vital information to formulate a preparation with adequate consistency and strength. The swelling of the Pluronic™ gels is characterized by two methods: (1) Monitor the dimensional changes as a function of immersion time under a constant load using a thermomechanical analyzer (TMA). PGG-polymer formulations are transferred into 5 mL beakers to a fixed height, taking care to avoid the introduction of any air bubbles. The analytical probe (10 mm diameter ebonite cylinder) is compressed into each sample at a definite rate (1 mm/s) to a depth of 15 mm and then retraced through its original path. The acquisition parameters are 5 mm/s pre-contact, 1 mm/s test speed, 10 mm/s post-contact with an acquisition rate of 50 points/sec using a 5 kg load cell. The resulting profiles are analyzed for firmness, cohesiveness and consistency of all gel formulations. Qualitative changes in Young's modulus are also determined to predict changes in mechanical properties of the vehicle undergoing sol-gel transition. The Young's and elastic moduli of air dried and fully hydrated samples, bioadhesion, and cohesiveness are measured. (2) Monitor weight change in phosphate buffered saline (PBS, pH 7.4). The swelling experiments are performed in PBS at room temperature and also at 37° C. Air dried samples (M₀) are weighed and immersed either in 20 mL deionized water or in PBS buffer, and maintained at 48 hrs in a heated water bath. Excess fluids from swollen samples are then carefully removed and weight change (M-M₀) with respect to dry mass is recorded, so as to calculate percent change in mass during swelling.

### In Vitro PGG Release Study

These tests serve as a comparative tool during the development of in vivo gel formulation. The release of PGG from the gel is studied by two compartment diffusion cell. In this system cellulose membrane (molecular weight cutoff=3000; Spectrapore) is separating the gel (2.0 g) containing PGG in the donor compartment with phosphate buffered saline (H 7.4, 37° C., 150 mL) in the receptor compartment. The effective diffusion area is 3.8 cm². The release data of PGG is plotted against the square root of time equation. Samples are withdrawn at various time intervals and analyzed by UV spectrophotometer at λₘₐₓ of 289 nm. The in vitro release profiles optimize several parameters like drug-to-polymer ratio, drug loading, and kinetics of drug release.

### Morphological Characterization of Degraded Nanoparticles

Nanoparticle degradation is monitored using an environmental scanning electron microscope (ESEM). Experiments are done on prepared nanoparticles and hydrogel dispersed nanoparticles. Their morphology is compared at various intervals over a 4 week study period.

### Prophetic Example 3

### In Vivo PGG Release and Organ Distribution (Table 2)

Once two release formulations that would deliver PGG for 24 hours (Pluroric™ hydrogel only) and sustained release for 28 days (PLGA nanoparticles dispersed in Pluronic™ hydrogel) are optimized, their application are tested in vivo.

### AAA Model

Aneurysms are induced in the abdominal aorta of 36 adult male Sprague-Dawley rats (˜250 g) using perivascular application of calcium chloride (CaCl₂) as originally described by Gertz et al. in J Clin Invest 1988;81(3):649-656 entitled “Aneurysm of the rabbit common carotid artery induced by periarterial application of calcium chloride in vivo”, with minor modifications outlined by Vyavahare et al. in Circulation 2007;115(13):1729-1737 entitled “Elastin stabilization for treatment of abdominal aortic aneurysms.” and in Circulation 2004;110(22):3480-3487 entitled “Elastin degradation and calcification in an abdominal aorta injury model: role of matrix metalloproteinases”, all incorporated herein by reference. Following the gauze-mediated application of CaCl₂, the aorta is flushed with warm sterile saline.

### Preparation and Application of Pluronic™ Hydrogels

Radio labeled (³H) PGG (100 μg per gel or animal; details below) is incorporated into one of two groups: (1) Pluronic™ hydrogels (n=12), or (2) Pluronic™ hydrogels+PLGA nanoparticles (n=12). In the latter case, PGG is loaded onto PLGA nanoparticles, which is then dispersed within the Pluronic™ solution. As controls (n=12), rat aortas are treated with CaCl₂ and subjected to no further treatment. Once the rat abdominal aortas have been exposed and treated with CaCl₂, one of the two PGG-Pluronic™ formulations are applied as a solution (with the exception of controls) and localized to the abdominal aorta. Upon warming to body temperature, these formulations gel around the tissue in situ. Once the Pluronic™ solutions have fully gelled, the abdominal wall is sutured and the skin incision sutured and stapled. Rats are allowed to recover and maintained in standard conditions. At 28 days post-surgery, all rats from both groups are anesthetized, the abdominal aorta is re-exposed and cleaned of adhesions. Rats are then euthanized by CO₂ asphyxiation and aorta recovered for analysis.

TABLE 2
In vivo study showing Animal Groups and Time Line

| | (³H-PGG)- | (³H-PGG)-Pluronic + | | |
|---|---|---|---|---|
| Time line | Pluronic | nanoparticles | Controls | End point analysis |
| Day 0 | Perivascular CaCl₂ aortic injury (15 min with gauze); rinse | | | Measure diameter |
| | with saline | | | |
| Day 0 | Apply pluronic + | Apply pluronic solution | No further | Close animal; allow to |
| | PGG solution; | with PGG-loaded | action | recover |
| | allow to gel | nanoparticles; allow to gel | | |
| Day 28 | Euthanize; excise | Euthanize; excise tissues | No further | Quantify tritium |
| | tissues | | action | within aorta and |
| | | | | surrounding tissues |
| Rats | 12 | 12 | 12 | Total rats 36 |

### Radio Labeled PGG

PGG is labeled with tritium (³H), a radioactive compound that can be easily quantified with a liquid scintillation counter. PGG is sent to and labeled by American Radiolabeled Chemicals, Inc. (St. Louis, Mo.), a company which specializes in such customized labeling. Abdominal aortic samples are collected 28 days after surgery (and initial delivery of the (³H-PGG)-polymer formulation) and analyzed for radioactivity. Once excised, the tissues are washed in buffered saline overnight, then digested in Solvable (Perkin-Elmer, Inc.; Wellesley, Mass.), a commercial preparation of sodium hydroxide formulated to not interfere with liquid scintillation. These digests then are to be diluted in liquid scintillation fluid (Hionic-Fluour, Perkin-Elmer, Inc.) and measured for tritium content. In addition to quantifying ³H-PGG within the primary area of interest, the abdominal aorta, the distribution of PGG throughout other neighboring tissues and organs are also analyzed. Insight as to how well the polymer formulations localized the release of PGG, and potentially what effect any “leaching” of PGG might have had is shown. Tritium is also quantified within excised thoracic aorta, heart, lungs, liver, and kidneys, using the digestion and quantification methods described above for abdominal aorta.

### Prophetic Example 4

### Treat Aneurysms with Perivascular Application of PGG through Pluronic™ Hydrogel or Polymeric Microparticles

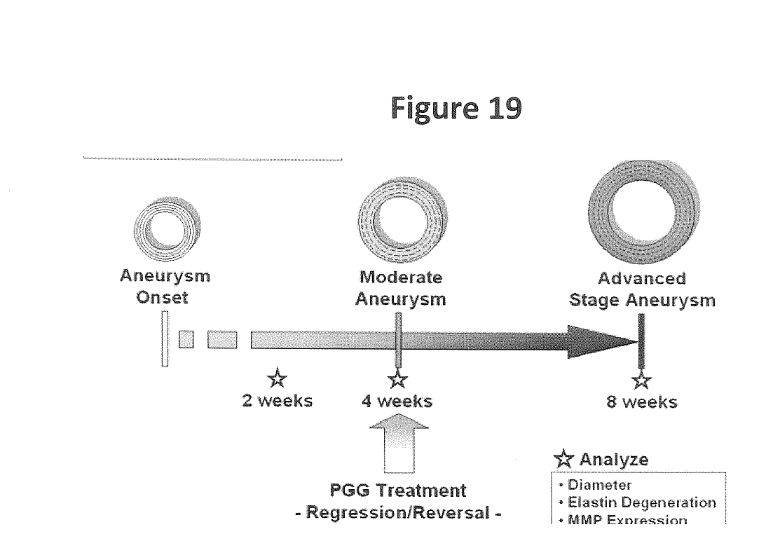

The efficacy of the aforementioned polymer delivery vehicles to administer PGG and retard or inhibit AAA progression in rats is tested. The hallmarks of AAAs are MMP-mediated elastin degeneration, dramatic changes in vascular architecture, structural weakening, dilatation and eventual rupture of the aorta. With its ability to reduce the susceptibility of elastin towards enzymatic degeneration, PGG has shown great promise in limiting AAA progression. The in vivo efficacy of PGG is evaluated when administered by clinically relevant polymer-based delivery vehicles: one which delivers PGG in a quick bolus-like dosage, while the other delivers PGG progressively over the course of 28 days in rats. This experiment more closely reflects the clinical situation, where early or moderate stage aneurysms could be stabilized by PGG application. As shown in experimental approach outlined in FIG. 19, AAA formation is induced in rats and the efficacies of two different polymer-based delivery vehicles for PGG application are tested. These delivery vehicles (Pluronic™ hydrogel and polymeric microparticles) are compared and investigated for their ability to administer PGG and the subsequent effect on aneurysm progression. PGG is applied weeks after CaCl₂ mediated aortic injury, so that the PGG treatment is administered to moderately aneurysmal aorta. The time-dependent diameter expansion as compared to vehicle-treated controls is monitored and the major features of AAA, specifically aortic elastin integrity, MMP activities and infiltration of host cells are analyzed.

### AAA Model

Aneurysms are induced in the abdominal aorta of 48 adult male Sprague-Dawley rats (˜250 g) using the protocol outlined in the prophetic example 3.

### Application of PGG Delivery Vehicles to Aneurysmal Aorta (Table 3)

The infrarenal abdominal aorta will be exposed by laparatomy through a midline incision, aortic diameter is measured by digital photography, and aorta treated periadventitially by applying a 15×5 mm, 0.5 M CaCl₂-presoaked, 8-ply piece of sterile gauze on the anterior surface of the aorta for 15 minutes, followed by 3 brief rinses with warm sterile saline. Incisions are closed and rats are allowed to recover. Subsequent treatments of PGG-polymer formulations (or, as controls, polymer vehicles alone) are administered at 28 days post-surgery, so as to be treating aortas which are already aneurysmal. At day 28, abdominal aorta is re-exposed, aortic diameter measured, and then treated with one of four groups described below. For Group A, (PGG-Pluronic™ treatment; n=12 rats), a solution of Pluronic™ acid and PGG with a predetermined concentrations and ratios is administered at the aneurysmal site (site of CaCl₂ treatment). This Pluronic™ solution is formulated to gel at 37° C., the approximate temperature encountered in vivo. Rats from Group B (PGG-Pluronic™+nanoparticles; n=12) are similarly treated, but with PGG loaded into PLGA nanoparticles (optimal conditions derived from previous examples), and the nanoparticles contained with the Pluronic™ solution. Groups C and D (Pluronic™ vehicle and Pluronic™+nanoparticles vehicle, respectively; n=12) serve as vehicle-only controls. In the case of all groups, once the Pluronic™ solutions have fully gelled around the aorta, the abdominal wall is sutured and the skin incision sutured and stapled. Rats are allowed to recover and maintained in standard conditions for another 28 days. At 56 days post surgery (28 days after PGG application), rats from each group are anesthetized, the abdominal aorta re-exposed, cleaned of adhesions, and photographed for diameter measurements. Rats are then euthanized by CO₂ asphyxiation and aorta recovered for analysis.

TABLE 3
Rat study showing Animal Groups and Time Line

| | | | | Group D | |
|---|---|---|---|---|---|
| | Group A | Group B | Group C | Pluronic + | |
| | PGG- | PGG-Pluronic + | Pluronic | nanoparticles | |
| Time line | Pluronic | nanoparticles | (vehicle only) | (vehicle only) | End point analysis |
| Day 0 | Perivascular CaCl₂ aortic injury (15 min); rinse with saline | | | | Measure diameter |
| Day 28 (2nd | Re-expose abdominal aorta; apply respective treatment | | | | Measure diameter |
| surgery) | | | | | |
| Day 56 | Aorta (12 rats) | Aorta (12 rats) | Aorta (12 rats) | Aorta (12 rats) | Diameter, elastin |
| | | | | | degeneration, MMP |
| | | | | | activities, inflammation |
| Rats | 12 | 12 | 12 | 12 | Total rats 48 |

### Aorta Retrieval and Analysis

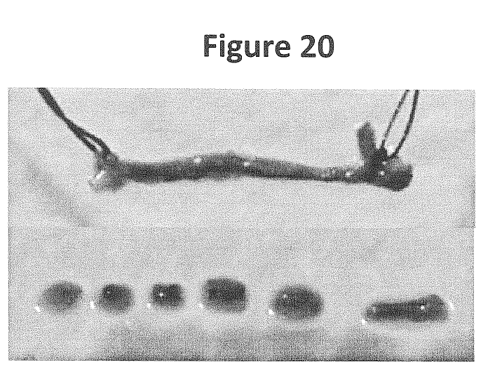

Measuring aortic diameters are done by digital photography before euthanasia. After euthanasia, the abdominal aorta is excised and divided into segments as shown in FIG. 20 for analysis: two segments are immediately frozen on dry ice for extraction of elastin peptides and zymography and for desmosine/hydroxyproline assays, one segment is embedded in OCT for immunohistochemistry and histology, and one is fixed in Karnowsky's fixative for TEM.

### Evaluation of Elastin Degeneration and MMP Activities and Detection of Soluble Elastin-Peptides

Tissues is extracted in a Guanidine buffer, dialyzed, and centrifuged. Supernatants are analyzed for the presence of elastin-peptides by an ELISA method outlined by Lee et al. in Am J Pathol 2006;168:490-498, entitled “Mechanisms of elastin calcification in the rat subdermal model: Gene expression associated with elastin degradation and ectopic osteogenesis.”, incorporated herein by reference. These extracts are also used for gelatin zymography outlined by Vyavahare et al. in Cardiovasc Pathol 2004;13(3):146-155 entitled “Involvement of matrix metalloproteinases and tenascin-C in elastin calcification.”, incorporated herein by reference, to evaluate MMP-2 and MMP-9 activities. Samples for desmosine/hydroxyproline analysis are lyophilized, weighed, hydrolyzed in HCl, and analyzed by radioimmunoassay outlined by Basalyga et al. in Circulation 2004;110(22):3480-3487 entitled “Elastin degradation and calcification in an abdominal aorta injury model: role of matrix metalloproteinases”, incorporated herein by reference. Calcium quantification is done by atomic absorption spectrophotometry on these same acid hydrolysates using the method outlined by Vyavahare et al. in Am J Pathol 1999;155(3):973-982 entitled “Elastin calcification and its prevention with aluminum chloride pretreatment.”, incorporated herein by reference.

### Histology and Ultra Structure

Retrieved aorta is stained with Hematoxylin and Eosin (H&E) for general structure, Verhoeff van Giesson for elastin, and with Alizarin Red for calcium deposits using methods outlined by Vyavahare et al. in Am J Pathol 2000;157(3):885-893 entitled “Inhibition of matrix metalloproteinase activity attenuates tenascin-C production and calcification of implanted purified elastin in rats.”, incorporated herein by reference. Appropriate timing for delayed PGG application demand careful evaluation. This timing could be reiterated based on results obtained from prophetic Example 3. A 25-50% increase in aortic diameter can be intervened with the second surgery.

### Statistics for Research Design and Methods

Variances computed from previous preliminary studies were used to design the prophetic experiments outlined herein to demonstrate the feasibility of using PGG for aneurysm treatment in vivo with applicable delivery methods. The sample size (n=6 for most in vitro studies, n=12 for in vivo studies) was chosen so that alpha (the probability of falsely claiming a difference) is 0.01, that beta (the probably of falsely claiming no difference) is 0.01, and that these conditions are met in the event of occasional loss of experimental samples. Variance analysis for a completely random design is done for all data and the means using least significant difference (LSD) is compared.

We claim:

1. A therapeutic composition for treatment of aneurysm in a patient, the therapeutic composition comprising connective tissue stabilization agent in combination with a delivery vehicle, wherein the delivery vehicle comprises a hydrogel, nanoparticles, or a combination thereof.

2. The therapeutic composition of claim 1 wherein the hydrogel comprises penta-galloylglucose in a gel form.

3. The therapeutic composition of claim 1 wherein the hydrogel comprises Pluronic™ hydrogel.

4. The therapeutic composition of claim 1 wherein the hydrogel, the nanoparticle, or both is or are loaded with penta-galloylglucose, glutaraldehyde, or a combination thereof.

5. The therapeutic composition of claim 1 wherein the nanoparticles comprises poly(lactic acid-co-glycolic) acid.

6. The therapeutic composition of claim 1 wherein the connective tissue stabilization agent comprises an elastin stabilization agent, a collagen stabilization agent, or a combination thereof.

7. The therapeutic composition of claim 6 wherein the elastin stabilization agent comprises a hydrophobic region and a plurality of functional groups capable of hydrogen bonding.

8. The therapeutic composition of claim 7 wherein the elastin stabilization agent comprises tannic acid or a derivative thereof, a flavonoid or a flavonoid derivative, a flavolignan or a flavolignan derivative, a phenolic rhizome or a phenolic rhizome derivative, a flavan-3-ol or a flavan-3-ol derivative, an ellagic acid or an ellagic acid derivative, a procyanidin or a procyanidin derivative, anthocyanins, quercetin, (+)-catechin, (−)epicatechin, pentagalloylglucose, nobotanin, epigallocatechin gallate, gallotannins, an extract of olive oil or a derivative of an extract of olive oil, cocoa bean or a derivative of a cocoa bean, camellia or a derivative of camellia, licorice or a derivative of licorice, sea whip or a derivative of sea whip, aloe vera or a derivative of aloe vera, chamomile or a derivative of chamomile, a combination thereof, or a pharmaceutically acceptable salt thereof.

9. The therapeutic composition of claim 6 wherein the collagen stabilization agent comprises a cross-linker of functional groups in collagen.

10. The therapeutic composition of claim 6 wherein the collagen stabilization agent comprises glutaraldehyde, genipin, acyl azide, epoxyamine, a combination thereof, or a pharmaceutically acceptable salt thereof.

11. The therapeutic composition of claim 6 wherein the connective tissue stabilization agent further comprises gallic acid scavenger, a lipid lowering medication, an anti-bacterial agent, an anti-fungal agent, or a combination thereof.

12. A method of making a therapeutic composition for treatment of aneurysm in a patient, the method comprising,
combining a connective tissue stabilization agent with a delivery vehicle to form the therapeutic composition so the connective tissue stabilization agent is released over a period of time to the aneurysm upon contact with bodily fluids, wherein the delivery vehicle comprises a hydrogel, nanoparticles, or a combination thereof.

13. The method of claim 12 wherein the combining step comprises forming a solution of precursor of the hydrogel and the connective tissue stabilization agent.

14. The method of claim 12 wherein the combining step comprises forming a solution of Pluronic™ block copolymers with penta-galloylglucose, glutaraldehyde, or a combination thereof.

15. The method of claim 12 wherein the combining step comprises embedding the connective tissue stabilization agent into the nanoparticles.

16. The method of claim 15 wherein the connective tissue stabilization agent is embedded inside nanoparticles using emulsion solvent evaporation technique.

17. The method of claim 15 wherein combining step further comprises combining the connective tissue stabilization agent embedded nanoparticles with the hydrogels.

18. The method of claim 12 wherein the combining step comprises forming a dispersion of Pluronic™ block copolymers with penta-galloylglucose-loaded poly(lactic acid-co-glycolic) acid nanoparticles.

19. The method of claim 18 wherein combining step further comprises adding glutaraldehyde-loaded poly(lactic acid-co-glycolic) acid nanoparticles to the dispersion.

20. The method of claim 12 wherein the connective tissue stabilization agent comprises an elastin stabilization agent, a collagen stabilization agent, or a combination thereof.

21. The method of claim 12 wherein the therapeutic composition further comprises pharmaceutically acceptable carriers and/or excipients.

22. A method of using a therapeutic composition for the treatment of aneurysm in a patient, the method comprising,
applying the therapeutic composition to the aneurysm,
wherein the therapeutic composition comprises a connective tissue stabilization agent with a delivery vehicle, the connective tissue stabilization agent being released over a period of time to the aneurysm through the delivery vehicle and
wherein the delivery vehicle comprises a hydrogel, nanoparticles, or a combination thereof.

23. The method of claim 22 wherein the therapeutic composition is applied intravascular, perivascularly, or a combination thereof to the aneurysm.

24. The method of claim 22 further comprising isolating the aneurysm from within a blood vessel using a device placed within the blood vessel and aspirating the isolated aneurysm before the application of the therapeutic composition using the device.

25. The method of claim 22 wherein the therapeutic composition is applied to the aneurysm through a perivascular wrap.

26. The method of claim 22 wherein the connective tissue stabilization agent is collagen stabilization agent, elastin stabilization agent, or a combination thereof.

27. The method of claim 22 wherein the treatment is applied plurality of times to the aneurysm in the patient.

28. A method for treatment of aneurysm in a patient comprising applying connective tissue stabilization agent in the form of a hydrogel, nanoparticles, or a combination thereof to the aneurysm.

29. The method of claim 28 wherein the connective tissue stabilization agent is pentagalloylglucose, epigallocatechin gallate, or a combination thereof.

30. An active agent delivery vehicle comprising a hydrogel and nanoparticles dispersed within the hydrogel, wherein the nanoparticles comprise the active agent and a bioresorbable polymer binder.
